(19) United States
(12) Patent Application Publication
(10) Pub. No.: US 2026/0077021 A1
(43) Pub. Date: Mar. 19, 2026

(54) Compositions and Methods for Treating Endocrine Diseases and Disorders

(71) Applicant: Olfactive Al, Inc., Mountain View, CA (US)

(72) Inventors: Christopher Hanson, Mountain View, CA (US); William Harries, Colden, NY (US); Bradley Brown, San Mateo, CA (US)

(21) Appl. No.: 19/395,051

(22) Filed: Nov. 20, 2025

Related U.S. Application Data
(63) Continuation-in-part of application No. 18/892,760, filed on Sep. 23, 2024.
Application No. 18/892,760 is a division of application No. 18/615,150, filed on Mar. 25, 2024, now Pat. No. 12,102,664.
Application No. 18/615,150 is a continuation-in-part of application No. 18/430,796, filed on Feb. 2, 2024, now Pat. No. 12,115,134.
(60) Provisional application No. 63/615,100, filed on Dec. 27, 2023.

(51) Int. Cl. A61K 38/26; A61K 31/085; A61K 31/202

(52) U.S. Cl. CPC A61K 38/26; A61K 31/085; A61K 31/202

(57) ABSTRACT

Disclosed herein are compounds and ligands, and compositions formed therewith, which modulate insulin secretion, suppress appetite, and reduce body mass by activating G-protein coupled receptors (GPCRs), such as ectopic olfactory receptors, and transient receptor potential (TRP) ion channels. Also disclosed herein are methods for using the compositions to treat endocrine diseases, such as type-2 diabetes and obesity.

### CROSS-REFERENCES TO RELATED APPLICATIONS

This application is a continuation-in-part of U.S. application Ser. No. 18/892,760, filed on Sep. 23, 2024, which is a divisional of U.S. application Ser. No. 18/615,150, filed on Mar. 25, 2024, now U.S. Pat. No. 12,102,664, which is a continuation-in-part of U.S. application Ser. No. 18/430,796, filed on Feb. 2, 2024, now U.S. Pat. No. 12,115,134, which claims the benefit of U.S. Provisional Application No. 63/615,100, filed on Dec. 27, 2023.

### FIELD OF THE INVENTION

The present invention relates to compositions and methods for treating endocrine disorders. More particularly, the present invention relates to compositions and methods for treating endocrine disorders associated with abnormal insulin secretion and excessive body mass; particularly, obesity and diabetes mellitus, by modulating ectopic olfactory activity.

### BACKGROUND OF THE INVENTION

As is well established, an individual that presents with a body mass index (BMI) of greater than or equal to 30 kg/m² is typically deemed obese.

As is also well established, obesity is a disorder that affects the health of millions of adults and youth in the U.S. According to a 2021 Centers for Disease Control and Prevention (CDC) survey, the prevalence of obesity in the U.S. between 2017-2020 was 41.9% in adults, 9.2% of which being severely obese (i.e., adults having a BMI greater than or equal to 40 kg/m²), and 19.7% in youth.

Based on earlier CDC data, the above-noted prevalence of adult obesity increased approximately 10% over a ten (10) year period, and the prevalence of youth obesity increased approximately 7% during the same period.

The increasing prevalence of obesity is also a growing U.S. national security concern due to difficulties maintaining operational readiness among current servicemembers and shrinking recruitment pools. An October 2023 study by the American Security Project (ASP) reflects that military obesity rates across active duty personal increased by approximately 11.2% between 2012 and 2022, according to an October 2023 study. The study also reflects that 68% of active duty servicemembers are either overweight or obese, and that eating disorders in the military also increased by approximately 79% between 2017 and 2021.

The rapidly increasing prevalence of obesity is not limited to the U.S. Indeed, the increasing prevalence of obesity is generally regarded as an epidemic worldwide.

In addition to a reduced life expectancy compared to non-obese individuals, and the public stigma and discrimination associated with obesity, obese individuals also often present with diabetes mellitus.

Indeed, the International Diabetes Federation (IDF) reported that in 2021 alone over 300 million obese individuals worldwide were afflicted with diabetes mellitus.

Diabetes mellitus is generally characterized by hyperglycemia associated with abnormal insulin secretion, i.e., insufficient insulin production by the pancreas or insulin resistance exhibited by endogenous cells.

As is well established, in most instances, insulin secretion is induced by pancreatic β-cells when glucagon-like peptide-1 (GLP-1) binds to and activates GLP-1 receptor proteins on endogenous gastrointestinal (GI) cells, such as enteroendocrine L-cells.

In addition to inducing insulin secretion, it has been found that GLP-1 also decreases the rate of gastric emptying and acid secretion, resulting in reduced appetite and, thereby, weight loss.

Individuals that present with unacceptable body mass and, hence, obesity are typically difficult to treat due to long term unhealthy eating patterns and a myriad of physiological complexities associated with the mechanisms of appetite control and energy metabolism. Treatment of such individuals, thus, typically requires significant adjustments in food consumption, and pharmaceutically active agents that increase insulin secretion and/or suppress the appetite of the individual.

Various entities have thus developed pharmaceutically active agents and therapies to modulate GLP-1 activity, i.e., activate GLP-1 receptor proteins on endogenous gastrointestinal (GI) cells, to treat individuals that present with diabetes mellitus and obesity.

Such pharmaceutically active agents include semaglutide (Ozempic®, Rybelsus®, Wegovy®), dulaglutide (Trulicity®), exenatide (Bydureon BCise®, Byetta®), and liraglutide (Victoza®, Saxenda®).

The noted pharmaceutically active agents (referred to hereinafter as “GLP-1 analogs”) mimic endogenous GLP-1 and are adapted to activate the GLP-1 receptors on endogenous GI cells and, hence, function as GLP-1 receptor agonists.

Although the GLP-1 analogs can effectively activate GLP-1 receptors on pancreatic β-cells and, hence, can induce insulin secretion and suppress the appetite of an individual, there are several drawbacks and disadvantages associated with administration of the GLP-1 analogs to patients.

A major drawback associated with administration of the GLP-1 analogs to patients is the high risk of adverse pathological events. One such adverse pathological event is hypoglycemia (i.e., low blood glucose), which can, and often will, present in patients that are also taking or being administered commonly prescribed antidiabetic agents, such as basal insulin and sulfonylureas.

There is also a high risk of induced production of anti-GLP-1 antibodies and binding of endogenous GLP-1 and the GLP-1 analogs to the anti-GLP-1 antibodies, which can, and often will, induce adverse immune responses.

A further major drawback associated with administration of GLP-1 analogs to individuals are the significant side effects that are often presented by the individuals, including nausea, vomiting, diarrhea, abdominal pain, and constipation.

Since most GLP-1 analogs are administered to patients via a subcutaneous injection, a further drawback associated with GLP-1 analog administration is the pain and discomfort associated with the often-prescribed weekly injections.

Although the GLP-1 analogs developed by Novo Nordisk, which are marketed under the tradename Rybelsus, can also be delivered orally, a significantly greater dose of the Rybelsus GLP-1 analog must be orally administered to an individual to match the pharmacokinetics of the Novo Nordisk injectable GLP-1 analog, which is marketed under the tradename Ozempic, i.e., individuals must be orally administered approximately 100.0 mg/week of the Rybelsus GLP-1 analog to match the efficacy of the typically prescribed 0.5 mg/week of the injectable Ozempic GLP-1 analog.

A further major drawback associated with administration of GLP-1 analogs to individuals is the cost. Indeed, the costs, at present, for a thirty (30) day supply of Ozempic and Rybelsus are approximately $1000.00 and $1200.00, respectively.

As is also well established, in most instances, insulin secretion is also induced by pancreatic β-cells when gastric inhibitory polypeptide (GIP) binds to and activates GIP receptor proteins on the pancreatic β-cells.

Although GIP also induces insulin secretion, there are also several drawbacks and disadvantages associated with administration of GIP alone to patients to treat abnormal insulin secretion.

A major disadvantage is that GIP also induces glucagon secretion from pancreatic β-cells. Since glucagon is a hyperglycemic compound that increases blood sugar when secreted, the increase in glucagon secretion induced by GIP limits its therapeutic potential for treating abnormal insulin secretion.

To address the above noted disadvantage associated with solely activating the GIP receptor proteins on the pancreatic β-cells, Eli Lilly has recently developed a pharmaceutically active agent that targets GLP-1 and GIP receptors on pancreatic β-cells.

The noted pharmaceutically active agent comprises tirzepatide, i.e., a dual GLP-1/GIP analog marketed under the tradenames Mounjaro® and ZepBound®, which provides the beneficial metabolic activity induced by both GLP-1 and GIP in a synergistic manner without a clinically significant increase in glucagon secretion.

Although the dual GLP-1/GIP analogs can effectively activate both GLP-1 and GIP receptors on GI cells and, hence, can induce insulin secretion, many of the drawbacks and disadvantages associated with administration of the GLP-1 analogs alone to patients are also associated with administration of the dual GLP-1/GIP analogs to patients.

Such drawbacks and disadvantages include the significant side effects that are often presented by the individuals, including nausea, vomiting, diarrhea, abdominal pain, kidney problems, and constipation. Indeed, it has been found that approximately 10% of individuals administered dual GLP-1/GIP analogs suffer from the noted side effects.

A further drawback also associated with dual GLP-1/GIP analog administration to patients is the pain and discomfort associated with the often-prescribed weekly injections. Since there are currently no known dual GLP-1/GIP analogs that are approved by the FDA for oral administration, painful weekly subcutaneous injections are the only available route of administration.

A further major drawback that is similarly associated with administration of dual GLP-1/GIP analogs to individuals is the cost. Indeed, the costs, at present, for a thirty (30) day supply of Mounjaro and Zepbound are similarly approximately $1000.00.

There is thus a need for improved compositions and methods to modulate GLP-1 and GIP secretion, which substantially reduce or overcome the drawbacks and disadvantages associated with the conventional GLP-1 analogs and dual GLP-1/GIP analogs discussed above.

It is thus one object of the present invention to provide improved compositions and methods to modulate GLP-1 and GIP secretion, which overcome the drawbacks and disadvantages associated with administration of GLP-1 analogs and dual GLP-1/GIP analogs.

It is another object of the present invention to provide improved compositions and methods that can be administered to a subject that presents with abnormal insulin secretion, which effectively modulate GLP-1 and GIP receptor activity on pancreatic β-cells and, thereby, modulate systemic insulin secretion without the undesirable side effects associated with administration of GLP-1 analogs and dual GLP-1/GIP analogs.

It is another object of the present invention to provide compositions comprising natural compounds and ligands that can be administered to a subject that presents with abnormal insulin secretion and, thereby, diabetes mellitus, which effectively modulates the insulin secretion of the individual and effectively treats the diabetes mellitus when administered thereto.

It is another object of the present invention to provide improved compositions and methods that can be administered to a subject that presents with unacceptable body mass, which effectively modulate GLP-1 and GIP receptor activity on pancreatic β-cells and, thereby effectively suppress the appetite of the subject, without the undesirable side effects associated with administration of GLP-1 analogs and dual GLP-1/GIP analogs.

It is another object of the present invention to provide compositions comprising natural compounds and ligands that can be administered to a subject that presents with unacceptable body mass and, thereby, obesity, which effectively modulate GLP-1 and GIP receptor activity on pancreatic β-cells and, thereby effectively suppress the appetite of the subject when administered thereto.

It is another object of the present invention to provide improved compositions that effectively modulate the endocrine system of a subject with minimal side effects, which can be administered to the subject via oral (or enteric), sublingual, inhalation, intranasal, epidural, intracerebral, transdermal, topical, and injection administration means.

It is another object of the present invention to provide improved compositions and methods that effectuate olfactory receptor (OR)-mediated secretion of endogenous GLP-1 and PYY in vivo, without the undesirable side effects associated with administration of GLP-1 analogs and dual GLP-1/GIP analogs.

It is another object of the present invention to provide improved compositions and methods that effectuate OR-mediated secretion of endogenous GIP in vivo, without the undesirable side effects associated with administration of GLP-1 analogs and dual GLP-1/GIP analogs.

It is another object of the present invention to provide improved compositions and methods that effectuate OR-mediated secretion of endogenous GLP-1 and GIP in vivo, without the undesirable side effects associated with administration of GLP-1 analogs and dual GLP-1/GIP analogs.

It is another object of the present invention to provide improved compositions and methods that effectuate free fatty acid receptor-mediated secretion of endogenous GIP in vivo, without the undesirable side effects associated with administration of GLP-1 analogs and dual GLP-1/GIP analogs.

It is another object of the present invention to provide improved compositions and methods that effectuate transient receptor potential ion channel-mediated secretion of endogenous GIP in vivo, without the undesirable side effects associated with administration of GLP-1 analogs and dual GLP-1/GIP analogs.

It is another object of the present invention to provide improved compositions and methods that effectuate OR-mediated, free fatty acid receptor-mediated, and transient potential ion channel-mediated secretion of endogenous GLP-1, PYY and GIP in vivo, without the undesirable side effects associated with administration of GLP-1 analogs and dual GLP-1/GIP analogs.

It is another object of the present invention to provide improved compositions that can effectively and safely induce secretion of GLP-1 and/or PYY and/or GIP in vivo, which can be administered to an individual via oral (or enteric), sublingual, inhalation, intranasal, epidural, intracerebral, transdermal, topical, and injection administration means.

### SUMMARY OF THE INVENTION

The present invention is directed to compositions and methods for treating endocrine diseases and disorders, and underlying causes thereof.

In some embodiments of the invention, there are thus provided compositions and methods for treating endocrine disorders of a subject that are associated with excessive and, hence, unacceptable body mass.

In one embodiment of the invention, a method for reducing body mass of a subject comprises the following steps:
(a) providing a composition comprising (i) a delivery medium, (ii) at least a first natural receptor activating compound adapted to bind to and activate at least one first receptor selected from the group comprising olfactory receptor family 51 subfamily E member 1 (OR51E1) and olfactory receptor family 2 subfamily C member 1 (OR2C1), and (iii) at least a second natural receptor activating compound adapted to bind to and activate at least one second receptor selected from the group comprising free fatty acid receptor 1 (FFAR1) and free fatty acid receptor 4 (FFAR4),
the composition adapted to induce glucagon-like peptide-1 (GLP-1) and gastric inhibitory polypeptide (GIP) secretion, and, thereby, increased insulin secretion in vivo, when the composition is delivered to the subject; and
(b) delivering the composition to the subject, wherein the composition suppresses the appetite of the subject.

In a preferred embodiment, the first receptor activating compound comprises butyl butyryl lactate.

In a preferred embodiment, the butyl butyryl lactate comprises an EC₅₀ concentration of at least 0.1 μM in the composition.

In a preferred embodiment, the second receptor activating compound comprises a compound selected from the group consisting of a medium-chain free fatty acid, a long-chain free fatty acid and an omega-3 polyunsaturated fatty acid.

In a preferred embodiment, the second receptor activating compound comprises a medium-chain free fatty acid.

In a preferred embodiment, the medium-chain free fatty acid comprises lauric acid.

In a preferred embodiment, the lauric acid comprises an EC₅₀ concentration of at least 0.05 μM in the composition.

In some embodiments of the invention, the composition comprises at least a third receptor activating compound adapted to bind to and activate at least one third receptor selected from the group comprising olfactory receptor family 2 subfamily B member 11 (OR2B11), olfactory receptor family 2 subfamily J member 3 (OR2J3), and transient receptor potential cation channel subfamily A member 1 (TRPA1).

In a preferred embodiment, the third receptor activating compound comprises cinnamaldehyde.

In a preferred embodiment, the cinnamaldehyde comprises an EC₅₀ concentration of at least 0.1 μM in the composition.

In some embodiments of the invention, the composition comprises at least a fourth receptor activating compound adapted to bind to and activate olfactory receptor family 2 subfamily W member 1 (OR2W1).

In a preferred embodiment, the fourth receptor activating compound comprises benzyl acetate.

In a preferred embodiment, the benzyl acetate comprises an EC₅₀ concentration of at least 0.1 μM in the composition.

In another embodiment of the invention, a method for reducing body mass of a subject comprises the following steps:
(a) providing a composition comprising a delivery medium, a first receptor activating compound, a second receptor activating compound and a third receptor activating compound,
the composition comprising a mass in the range of approximately 1200.0 mg to approximately 1400.0 mg,
the delivery medium comprising in the range of approximately 55.0% to approximately 60.0% (w/w) of the composition,
the first receptor activating compound comprising butyl butyryl lactate, the butyl butyryl lactate comprising in the range of approximately 3.0% to approximately 5.0% (w/w) of the composition,
the second receptor activating compound comprising lauric acid, the lauric acid comprising in the range of approximately 0.1% to approximately 0.5% (w/w) of the composition,
the third receptor activating compound comprising cinnamaldehyde, the cinnamaldehyde comprising in the range of approximately 24.0% to approximately 26.0% (w/w) of the composition,
the composition adapted to induce activation of at least olfactory receptor family 51 subfamily E member 1 (OR51E1), free fatty acid receptor 1 (FFAR1) and transient receptor potential cation channel subfamily A member 1 (TRPA1) in vivo; and
(b) delivering the composition to the subject, wherein secretion of glucagon-like peptide-1 (GLP-1) and gastric inhibitory polypeptide (GIP) and, thereby, increased insulin secretion is induced.

In some embodiments, the composition further comprises a fourth receptor activating compound adapted to bind to and induce activation of at least OR51E1.

In some embodiments, the fourth receptor activating compound comprises eugenol.

In some embodiments, the eugenol comprises at least approximately 3.0% (w/w) of the composition.

In some embodiments, the composition further comprises a fifth receptor activating compound. adapted to bind to and induce activation of at least olfactory receptor family 2, subfamily W, member 1 (OR2W1).

In some embodiments, the fifth receptor activating compound comprises benzyl acetate.

In some embodiments, the benzyl acetate comprises at least approximately 0.5% (w/w) of the composition.

In some embodiments, the composition further comprises a sixth receptor activating compound adapted to bind to and activate olfactory receptor family 2 subfamily B member 11 (OR2B11).

In some embodiments, the sixth receptor activating compound comprises spearmint oil.

In some embodiments, the spearmint oil comprises at least approximately 7.0% (w/w) of the composition.

In some embodiments, the delivery medium comprises sunflower seed oil.

In a preferred embodiment, delivery of the composition to the subject comprises enteric sequential delivery of the composition to the subject via a dosage delivery protocol comprising first delivery of a first dose of the composition to the subject at a first time, second delivery of a second dose of the composition to the subject at a first period of time in the range of approximately 3.5 to approximately 4.5 hours after the first delivery of the first dose of the composition to the subject, third delivery of a third dose of the composition to the subject at a second period of time in the range of approximately 3.5 to approximately 4.5 hours after the second delivery of the second dose of the composition to the subject, and fourth delivery of a fourth dose of the composition to the subject at a third period of time in the range of approximately 3.5 to approximately 4.5 hours after the third delivery of the third dose of the composition to the subject, whereby at least activity of OR51E1, FFAR1 and TRPA1 is induced and sustained from the first time of the first delivery of the first dose of the composition to a fourth period of time in the range of approximately 3.5 to approximately 4.5 hours after the fourth delivery of the fourth dose of the composition to the subject.

In another embodiment of the invention, a method for reducing body mass of a subject, comprises the following steps:
(a) providing a composition comprising a delivery medium, a first receptor activating compound, a second receptor activating compound and a third receptor activating compound,
the composition comprising a mass in the range of approximately 600.0 mg to approximately 800.0 mg,
the delivery medium comprising in the range of approximately 79.0% to approximately 85.0% (w/w) of the composition,
the first receptor activating compound comprising butyl butyryl lactate, the butyl butyryl lactate comprising in the range of approximately 1.0% to approximately 3.0% (w/w) of the composition,
the second receptor activating compound comprising lauric acid, the lauric acid comprising in the range of approximately 0.05% to approximately 0.2% (w/w) of the composition,
the third receptor activating compound comprising cinnamaldehyde, the cinnamaldehyde comprising in the range of approximately 11.0% to approximately 13.5% (w/w) of the composition,
the composition adapted to induce activation of at least olfactory receptor family 51 subfamily E member 1 (OR51E1), free fatty acid receptor 1 (FFAR1) and transient receptor potential cation channel subfamily A member 1 (TRPA1) in vivo; and
(b) delivering the composition to the subject, wherein secretion of glucagon-like peptide-1 (GLP-1) and gastric inhibitory polypeptide (GIP) and, thereby, increased insulin secretion is induced.

In some embodiments, the composition further comprises a fourth receptor activating compound adapted to bind to and induce activation of at least OR51E1.

In some embodiments, the fourth receptor activating compound comprises eugenol.

In some embodiments, the eugenol comprises at least 1.0% (w/w) of the composition.

In some embodiments, the composition further comprises a fifth receptor activating compound. adapted to bind to and induce activation of at least olfactory receptor family 2, subfamily W, member 1 (OR2W1).

In some embodiments, the fifth receptor activating compound comprises benzyl acetate.

In some embodiments, the benzyl acetate comprises at least 0.1% (w/w) of the composition.

In some embodiments, the composition further comprises a sixth receptor activating compound adapted to bind to and activate olfactory receptor family 2 subfamily B member 11 (OR2B11).

In some embodiments, the sixth receptor activating compound comprises spearmint oil.

In some embodiments, the spearmint oil comprises at least 2.0% (w/w) of the composition.

In some embodiments, the delivery medium comprises sunflower seed oil.

In a preferred embodiment, delivery of the composition to the subject comprises enteric sequential delivery of the composition to the subject via a dosage delivery protocol comprising first delivery of a first dose of the composition to the subject at a first time, second delivery of a second dose of the composition to the subject at a first period of time in the range of approximately 2.5 to approximately 3.5 hours after the first delivery of the first dose of the composition to the subject, third delivery of a third dose of the composition to the subject at a second period of time in the range of approximately 2.5 to approximately 3.5 hours after the second delivery of the second dose of the composition to the subject, and fourth delivery of a fourth dose of the composition to the subject at a third period of time in the range of approximately 2.5 to approximately 3.5 hours after the third delivery of the third dose of the composition to the subject, whereby at least activity of OR51E1, FFAR1 and TRPA1 is induced and sustained from the first time of the first delivery of the first dose of the composition to a fourth period of time in the range of approximately 2.5 to approximately 3.5 hours after the fourth delivery of the fourth dose of the composition to the subject.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of receptor-mediated activation of GLP-1 secretion from endogenous gastrointestinal cells;

FIG. 2 is a schematic illustration of receptor-mediated activation of GIP secretion from endogenous gastrointestinal cells;

FIG. 3 is a bar graph depicting induced OR51E1 activity, expressed as luminescence emanating from cells in vitro, by butyl butyryl lactate at various concentrations;

FIG. 4 is a bar graph depicting induced FFAR1 activity, expressed as luminescence emanating from cells in vitro, by lauric acid at various concentrations;

FIG. 5 is a bar graph depicting induced FFAR1 activity, expressed as luminescence emanating from cells in vitro, by butyl butyryl lactate, lauric acid, and a mixture of butyl butyryl lactate and lauric acid at concentrations of 1500.0 μM;

FIG. 6 is a bar graph depicting induced OR51E1 activity, expressed as luminescence emanating from cells in vitro, by eugenol at various concentrations; and

FIGS. 7A-7D are graphs of four (4) participants in a study of physiological changes induced after administration of a composition of the invention, depicting the substantial weight loss at the three (3) week time point.

### DETAILED DESCRIPTION OF THE PREFERRED EMBODIMENT

Before describing the present invention in detail, it is to be understood that this invention is not limited to particularly exemplified compounds, compositions or methods, as such may, of course, vary. Thus, although a number of compounds, compositions and methods similar or equivalent to those described herein can be used in the practice of the present invention, the preferred compounds, compositions and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a”, “an” and “the” include plural referents unless the content clearly dictates otherwise. Thus, for example, reference to “an active agent” includes two or more such agents and the like.

### Definitions

The term “endocrine disorder”, as used herein, means and includes any physiological disorder associated with abnormal insulin secretion or body mass, including, without limitation, diabetes mellitus and obesity.

The terms “excessive body mass”, “unacceptable body mass” and “undesirable body mass” are used interchangeably herein and mean and include a body mass categorized as comprising BMI greater than or equal to 30 kg/m² and less than 30 kg/m². In some embodiments, the terms “excessive body mass”, “unacceptable body mass” and “undesirable body mass” thus mean and include a body mass categorized as comprising a BMI greater than or equal to 25 kg/m².

The term “endocrine factor”, as used herein, means and includes any molecular compound that is produced and secreted by endogenous cells and induces biological activity at a biological tissue site. The term “endocrine factor” thus means and includes, without limitation, glucagon-like peptide-1 (GLP-1), gastric inhibitory polypeptide (GIP), peptide Y-Y (PYY), ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, glucagon, enteroglucagon, galanin, leptin, motilin, amylin, neuropeptide Y (NPY), pancreatic polypeptide, substance P, oxyntomodulin, and somatostatin.

The terms “protein”, “peptide”, “polypeptide” and “polypeptide fragment”, as used interchangeably herein, mean, and include amino acid polymers residues of any length. The amino acid polymer can be linear or branched, comprise modified amino acids or amino acid analogs, and it can be interrupted by chemical moieties other than amino acids. The terms “protein”, “peptide”, “polypeptide” and “polypeptide fragment” also include amino acid polymers that have been modified naturally or synthetically by chemical intervention; by way of example, disulfide bond formation, glycosylation, lipidation, acetylation, phosphorylation, PEGylation or any other manipulation or modification, such as conjugation with a labeling or bioactive component.

The term “agonist”, as used herein, means, and includes any molecule which binds to a receptor on a cell, wherein the binding to the receptor can potentially lead to subsequent changes in the cell's functions. When an agonist binds to a sufficient number of receptors, the receptors can activate seminal processes in the cell.

The term “antagonist”, as used herein, means and includes a molecule, which binds to a receptor on a cell and inhibits the receptor from activating processes in the cell. The inhibition of the receptor can include competitive binding against agonists (when an antagonist is bound, agonists cannot bind to the receptor) and allosteric effects (when the antagonist binds, agonists can still bind the receptor, but cannot activate the receptor).

The term “olfactory receptor (OR)”, as used herein, means and includes an olfactory receptor that is a seminal component of the chemosensory organs responsible for olfaction. The term “olfactory receptor” as used herein, also means, and includes, trace amine associated receptors, vomeronasal receptors, formyl peptide receptors, membrane guanylyl cyclase, subtype GC-D receptors; and G-protein coupled receptors, such as G-protein coupled taste receptors. Olfactory receptors can also include hybrid receptors synthesized from the above-noted olfactory receptors.

The term “ectopic olfactory receptor”, as used herein, means and includes an olfactory receptor that is present in organs, tissue, and/or cells that is a seminal component of physiological processes outside of olfaction and, in some instances, indirectly involved with olfactory-mediated processes.

The term “free fatty acid receptor”, as used herein, means and includes a transmembrane cell surface receptor that is adapted and configured to bind to fatty acids and induce cell signaling processes in response to the binding of the fatty acids.

The term “transient receptor potential ion channel”, as used herein, means and includes a transmembrane ion channel that is adapted and configured to modulate ion entry into an endogenous cell, such as Ca²⁺ entry, and, thereby, induce cell signaling process when a compound or ligand binds to the ion channel.

The term “compound”, as used herein, means and includes any composition of matter comprising two or more chemical elements. According to the invention, in some instances, the terms “compound” and “ligand” are synonymous and used interchangeably herein.

The term “compound” thus means and includes, without limitation, 3-methylpentanoic acid, pentanoic acid, pentanol, 4-methylnonanoic acid, eugenol, farnesol, farnesyl thiosalicylic acid, acrolein, formalin, hydrogen peroxide, coumarin, dicyclohexyl disulfide, nonanoic acid, octanioic acid, 2-nonanoic acid, butyric acid, heptanoic acid, decanoic acid, tetradecanoic acid, trans-2-decenoic acid, tridecanoic acid, undecanoic acid, methyl eugenol, methyl salicylate, (+)-menthol, eugenyl acetate, 2,4-dinitrotoluene, 4-hydroxynonenal, hexanoic acid, 2-ethylhexanoic acid, 2-ethyl-3,5-dimethylpyrazine, pyrazine, dimethyl disulfide, methyl furfuryl disulfide, propanal, butyl butyryl lactate, isovaleric acid, propionic acid, 4-methylpentanoic acid, methanoic acid, octanoic acid, octanal, coumarin, helional, lilial, β-ionone, androstenone, androstadienone, caramel furanone, 3-phenyl propyl propionate, ethyl vanillin, 2-ethyl-fencol, N-amyl acetate, eugenol acetate, sandalwood, S-(−)-citronellol, (−)-citronellol, hydroxycitronellal, citral, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, heptanol, hexanol, hexyl acetate, 1-hexanol, 1-heptanol, 2-heptanone, octanol, 1-octanol, celery ketone, anis aldehyde, vanillin, guaiacol, hydroxymethylpentylcyclohexenecarboxaldehyde (lyral), thujopsene, allyl phenylacetate, allyl isothiocyanate, benzyl acetate, 3,4-hexanedione, cis-3-hexen-1-ol, quinoline, ethyl heptanoate, methyl octanoate, nonanal, 1-nonanol, 2-nonanol, 3-octanone, 3-nonanone, decyl aldehyde, (E)-non-2-enal 2-ethyl-3,5-dimethylpyrazine 3-methylbut-2-ene-1-thiol, (2E,6Z)-nona-2,6-dienalcitral, ethyl octanoate, p-mentha-8-thiol-3-one, β-myrcene, y-decalactone, (S)-(+)-carvone, dihydrojasmone, cinnamaldehyde, spearmint oil, coffee difuran, quinoline, butyl anthranilate 2,2-dithiodimethylenedifuran, ethyl hexanoate, limonene, α-terpineol, eugenol (3E,5Z)-undeca-1,3,5-triene, long-chain free fatty acids (e.g., palmitic acid and stearic acid), medium-chain free fatty acids (e.g., caproic acid (C6:0), caprylic acid (C8:0), capric acid (C10:0), and lauric acid (C12:0)), and omega-3 polyunsaturated fatty acids (e.g., alpha-linoleic acid, docosahexaenoic acid, and eicosatetraenoic acid).

The term “compound” also means and includes any composition of matter included in the Food and Drug Administration's (FDA's) generally recognized as safe (GRAS) database.

The terms “composition”, “formulation”, “olfactory composition” and “olfactory formulation”, as used interchangeably herein, mean and include any compound or combination of compounds that can interact with and modulate at least one olfactory receptor and/or ectopic olfactory receptor and/or free fatty acid receptor and/or transient receptor potential ion channel.

The terms “olfaction” and “olfactory reception”, as used interchangeably herein, mean and include the interaction of a composition (or formulation) with an olfactory receptor coupled to a cell signaling pathway. The composition can also be defined as an “odorant” and may be airborne (i.e., volatile) and/or in solution.

The terms “express” and “expression”, as used interchangeably herein, mean, and include the production of a protein product from the genetic information contained within a nucleic acid sequence.

The term “upregulation”, as used herein, means, and includes the increased production of a protein product from the genetic information contained within a nucleic acid sequence.

The term “downregulation”, as used herein, means, and includes the decreased production of a protein product from the genetic information contained within a nucleic acid sequence.

The terms “delivery” and “administration” are used interchangeably herein, and mean and include providing a composition (or formulation), through any method appropriate to deliver the composition (or formulation) to a subject. According to the invention, such administration means includes, without limitation, oral, sublingual, inhalation, intranasal, epidural, intracerebral, transdermal, topical, and injection administration means.

The term “EC₅₀”, as used herein, means, and includes the concentration of a substance (e.g., a compound or a drug), which, after delivery to a subject, induces at least 50% activation or enhancement of a biological process.

In some embodiments, the term “EC₅₀” refers to the concentration of agonist which, after delivery to a subject, induces a response halfway between the baseline and maximum response in an in vitro assay.

In some embodiments, the term “EC₅₀” refers to the concentration of a modulator (e.g., an agonist) which, after delivery to a subject, induces at least 50% activation of a receptor type, by way of example, an ectopic olfactory receptor.

The term “IC₅₀”, as used herein, means, and includes the concentration of a substance (e.g., a compound or a drug), which, after delivery, inhibits or attenuates at least 50% of a biological process.

In some embodiments, the term “IC₅₀” refers to the concentration of a modulator (e.g., an antagonist or inhibitor), which, after delivery, inhibits or attenuates at least 50% of receptor activity, e.g., at least 50% of an ectopic olfactory receptor activity.

The term “sustained”, as used herein in connection with induced receptor activity, means and includes continued activity of at least one biological process, such as, by way of example, induced activity of an ectopic olfactory receptor for a continuous period of time.

The term “comprise” and variations of the term, such as “comprising” and “comprises”, means “including, but not limited to” and is not intended to exclude, for example, other compounds, ligands or method steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention.

As indicated above, the present invention is directed to compositions and methods for treating endocrine disorders associated with abnormal insulin secretion and body mass; particularly, diabetes mellitus and obesity, by modulating receptor activity.

As discussed above, various entities have developed GLP-1 analogs that mimic endogenous GLP-1 alone, and dual GLP-1/GIP analogs that mimic both endogenous GLP-1 and GIP in combination.

As also discussed above, the GLP-1 analogs activate the GLP-1 receptor on pancreatic β-cells and the dual GLP-1/GIP analogs activate both GLP-1 receptor and GIP receptor on pancreatic β-cells to modulate insulin secretion by the pancreas.

Although the GLP-1 analogs and dual GLP-1/GIP analogs can effectively modulate insulin secretion, as also discussed in detail above, there are several drawbacks and disadvantages associated with administration of GLP-1 analogs and dual GLP-1/GIP analogs to patients, including, a high risk of hypoglycemia, adverse side effects, and high costs.

As discussed in detail below, Applicant has developed compositions that directly and effectively modulate production of endogenous GLP-1 and GIP in vivo, which overcome the drawbacks and disadvantages associated with GLP-1 analogs that merely mimic endogenous GLP-1, and dual GLP-1/GIP analogs that merely mimic endogenous GLP-1 and GIP.

Although the compositions of the invention are described in connection with the treatment of endocrine diseases and disorders, and underlying causes thereof. As will readily appreciated by one having ordinary skill in the art, the compositions can also be employed to effectively treat additional diseases and/or disorders, including, without limitation, cardiovascular diseases and disorders, reproductive diseases and disorders, immune diseases and disorders, etc.

As discussed in detail below, in preferred embodiment, the compositions of the invention comprise at least one compound or ligand that is adapted to bind to and activate at least one receptor, e.g., an ectopic olfactory receptor and/or free fatty acid receptor and/or transient receptor potential ion channel, whereby insulin secretion is increased in vivo, and appetite is suppressed.

According to the invention, suitable compounds and ligands (also referred to herein as “receptor activating compounds and ligands”) include, without limitation, 3-methylpentanoic acid, pentanoic acid, pentanol, 4-methylnonanoic acid, eugenol, farnesol, farnesyl thiosalicylic acid, acrolein, formalin, hydrogen peroxide, coumarin, dicyclohexyl disulfide, nonanoic acid, octanioic acid, 2-nonanoic acid, butyric acid, heptanoic acid, decanoic acid, tetradecanoic acid, trans-2-decenoic acid, tridecanoic acid, undecanoic acid, methyl eugenol, methyl salicylate, (+)-menthol, eugenyl acetate, 2,4-dinitrotoluene, 4-hydroxynonenal, hexanoic acid, 2-ethylhexanoic acid, 2-ethyl-3,5-dimethylpyrazine, pyrazine, dimethyl disulfide, methyl furfuryl disulfide, propanal, butyl butyryl lactate, isovaleric acid, propionic acid, 4-methylpentanoic acid, methanoic acid, octanoic acid, octanal, coumarin, helional, lilial, β-ionone, androstenone, androstadienone, caramel furanone, 3-phenyl propyl propionate, ethyl vanillin, 2-ethyl-fencol, N-amyl acetate, eugenol acetate, sandalwood, S-(−)-citronellol, (−)-citronellol, hydroxycitronellal, citral, S-(−)-citronellal, (+)-carvine, (−) carvone, (+) carvone, linalool, bourgeonal, acetophenone, amyl butyrate, nonanethiol, allyl phenyl acetate, N-amyl acetate, muscone, isoeugenol, eugenol methyl ether, heptanol, hexanol, hexyl acetate, 1-hexanol, 1-heptanol, 2-heptanone, octanol, 1-octanol, celery ketone, anis aldehyde, vanillin, guaiacol, hydroxymethylpentylcyclohexenecarboxaldehyde (lyral), thujopsene, allyl phenylacetate, allyl isothiocyanate, benzyl acetate, 3,4-hexanedione, cis-3-hexen-1-ol, quinoline, ethyl heptanoate, methyl octanoate, nonanal, 1-nonanol, 2-nonanol, 3-octanone, 3-nonanone, decyl aldehyde, (E)-non-2-enal 2-ethyl-3,5-dimethylpyrazine 3-methylbut-2-ene-1-thiol, (2E,6Z)-nona-2,6-dienalcitral, ethyl octanoate, p-mentha-8-thiol-3-one, β-myrcene, γ-decalactone, (S)-(+)-carvone, dihydrojasmone, cinnamaldehyde, spearmint oil, coffee difuran, quinoline, butyl anthranilate 2,2-dithiodimethylenedifuran, ethyl hexanoate, limonene, α-terpineol, eugenol (3E,5Z)-undeca-1,3,5-triene, long-chain free fatty acids (e.g., palmitic acid and stearic acid), medium-chain free fatty acids (e.g., caproic acid (C6:0), caprylic acid (C8:0), capric acid (C10:0), and lauric acid (C12:0)), and omega-3 polyunsaturated fatty acids (e.g., alpha-linoleic acid, docosahexaenoic acid and eicosatetraenoic acid).

According to the invention, the receptor activating compounds and ligands (and, hence, compositions of the invention formed therefrom) are adapted to bind to and activate one or more of the following receptors: adipose olfactory receptors (e.g., OR51E2, OR2W3, OR51E1, OR2A1/42, OR2A4/7, OR52N4, OR13A1, 047D2, OR10J1, OR1L8, OR2B6, OR4D6, OLFR16, TAS1R3, TAS2R10, TAS2R13, TAS2R14, TAS2R19, TAS2R20, TAS2R31, TAS2R40, TAS2R42, TAS2R5, VN1R1, and VN1R2), adrenal olfactory receptors (e.g., OR51E2, ORW3, OR51E1, OR2A1/42, OR2A4/7, OR52N4, OR13A1, OR5K2, OR3A2, OR2H2, OR7C1, OR2L13, OR1L8, OR2T8, OR10AD1, OR52B6, OR1E1, OR13J1, OR2C1, OR52D1, OR10A2, OR2B6, OR8G5, OR1F12, OR4D6, TAS1R1, TAS1R3, TAS2R10, TAS2R13, TAS2R14, TAS2R19, TAS2R20, TAS2R3, TAS2R30, TAS2R31, TAS2R4, TAS2R42, TAS2R5, TAS2R50, TAS2R9, and VN1R1), central nervous system (CNS) olfactory receptors (e.g., OR51E2, OR2W3, OR4N4, OR51E1, OR52N4, OR13A1, OR5K2, OR7D2, OR3A2, OR2V1, OR2H2, OR7C1, OR2L13, OR1L8, OR2T8, OR10AD1, OR3A3, OR2K2, OR13J1, OR2C1, OR7A5, OR10A2, OR1F12, TAAR3, TAAR5, TAAR6. TAS1R1, TAS1R3, TAS2R1, TAS2R10, TAS2R13, TAS2R14, TAS2R19, TAS2R20, TAS2R3, TAS2R30, TAS2R31, TAS2R39, TAS2R4, TAS2R40, TAS2R42, TAS2R46, TAS2R5, TAS2R50, TAS2R7, TAS2R8, TAS2R9, VN1R1, VN1R2, and VN1R5), dopaminergic neuron olfactory receptors (e.g., OR51E1, OR51E2, and OR2J3), mammary olfactory receptors (e.g., OR51E2, OR51E1, OR2A1/42, OR2A4/7, OR52N4, OR5K2, OR3A2, OR2T8, OR10AD1, OR3A3, OR2K2, OR1E1, OR2C1, OR2C3, OR8D1, OR7A5, OR10A2, TAS1R1, TAS1R3, TAS2R10, TAS2R13, TAS2R14, TAS2R19, TAS2R20, TAS2R31, TAS2R4, TAS2R5, and VN1R1), cardiovascular olfactory receptors (e.g., OR51E2, OR51E1, OR52N4, OR13A1, OR2H2, OR10AD1, OR3A3, OR52B6, OR2K2, OR8G5, OR4D6, TAS1R1, TAS1R3, TAS2R10, TAS2R13, TAS2R14, TAS2R19, TAS2R20, TAS2R3, TAS2R30, TAS2R31, TAS2R4, TAS2R43, TAS2R46, TAS2R5, TAS2R50, TAS2R7, and VN1R1), renal olfactory receptors (e.g., OR51E2, OR51E1, OR2A1/42, OR2A4/7, OR5K2, OR1L8, OR10A2, OR1F12, TAS1R1, TAS1R3, TAS2R1, TAS2R10, TAS2R14, TAS2R19, TAS2R20, TAS2R3, TAS2R30, TAS2R31, TAS2R4, TAS2R42, TAS2R43, TAS2R5, TAS2R50, and VN1R1), hepatic olfactory receptors (e.g., OR2W3, OR51E1, OR2A1/42, OR2A4/7, OR7D2, OR1L8, OR2T8. TAS1R3, TAS2R14, TAS2R14, TAS2R20, TAS2R30, TAS2R30, TAS2R40, TAS2R5, VN1R1, and VN1R2), lymphatic olfactory receptors (e.g., OR51E2, OR51E1, OR2W3, OR2A1/42, OR2A4/7, OR52N4, OR13A1, OR5K2, OR7D2, OR3A2, OR2H2, OR3A3, OR2B6, OR52B6, TAS1R3, TAS2R14, TAS2R19, TAS2R20, TAS2R31, TAS2R4, TAS2R5, TAS2R40, TAS2R50, TAS2R43, TAS2R5, and VN1R1), ovarian olfactory receptors (e.g., OR51E2, OR2W3, OR4N4, OR51E1, OR2A1/42, OR2A4/7, OR52N4, OR5K2, OR3A2, OR2V1, OR2H2, OR2L13, OR1L8, OR10AD1, OR3A3, OR52B6, OR13J1, OR2C1, OR52D1, OR51B5, OR1F12, TAS1R1, TAS1R3, TAS2R1, TAS2R10, TAS2R13, TAS2R14, TAS2R19, TAS2R20, TAS2R3, TAS2R31, TAS2R4, TAS2R42, TAS2R43, TAS2R5, TAS2R50, TAS2R60, TAS2R7, VN1R1, and VN1R2), prostate olfactory receptors (e.g., OR51E2, OR2W3, OR51E1, OR2A1/42, OR2A4/7, OR52N4, OR13A1, OR5K2, OR2H2, OR7C1, OR1E1, OR13J1, OR51B5, TAS1R3, TAS2R14, TAS2R19, TAS2R20, TAS2R43, TAS2R46, TAS2R5, and VN1R1), dermal olfactory receptors (e.g., OR2AT4), testicular olfactory receptors (e.g., OR4N4, OR6F1, OR2H1, OR51E2, OR2W3, OR4N4, OR51E1, OR2A1/42, OR2A4/7, OR52N4, OR7D2, OR3A2, OR2V1, OR2H2, OR7C1, OR10J1, OR1L8, OR1C1, OR2H1, OR10AD1, OR3A3, OR13C3, OR2K2, OR1E1, OR2C1, OR2K2, OR1E1, OR2C1, OR2C3, OR8D1, OR52D1, OR7A5, OR10A2, OR2B6, OR7E24, OR6F1, OR8G5, OR51B5, OR1F12, TAS1R1, TAS1R3, TAS2R1, TAS2R14, TAS2R19, TAS2R20, TAS2R3, TAS2R31, TAS2R4, TAS2R43, TAS2R5, TAS2R50 TAS2R60, VN1R1, VN1R2, VN1R3, and VN1R4), hematologic olfactory receptors (e.g., OR2W3, OR2A4/7, OR52N4, OR7D2, OR2L13, OR3A3, OR2C1, OR2C3, OR2B6, TAS1R3, TAS2R14, TAS2R20, TAS2R40, and TAS2R60), trace amine-associated receptors (e.g., TAAR1, TAAR2, TAAR3, TAAR4P, TAAR5, TAAR6, TAAR7P, TAAR8, and TAAR9), gastrointestinal (GI) olfactory receptors (e.g., OR51E2, OR2W3, OR51E1, OR2A1/42, OR2A4/7, OR2C1, OR5K2, OR7D2, OR7C1, OR2L13, OR7A5, OR51B5, TAS1R1, TAS1R3, TAS2R14, TAS2R20, TAS2R4, TAS2R43, TAS2R5, and VN1R1, free fatty acid receptors (e.g., FFAR1 and FFAR4), and transient receptor potential ion channels (e.g., TRPA1).

According to the invention, the compositions of the invention can be administered to a subject or patient via oral (or enteric), sublingual, inhalation, intranasal, epidural, intracerebral, transdermal, topical, and injection administration means.

Thus, in some embodiments, the compositions of the invention are delivered enterically in tablet or capsule form.

In some embodiments of the invention, the receptor activating compounds and ligands of the invention referenced above and, hence, compositions of the invention formed therefrom are adapted to bind to and activate combinations of the aforementioned receptors, i.e., induce multiple receptor activity.

### Olfactory Receptor-Mediated GLP-1 and PYY Secretion

In some embodiments of the invention, the receptor activating compounds and ligands, and compositions of the invention formed therefrom are specifically adapted to bind to and activate at least one olfactory receptor including, without limitation, olfactory receptor family 51 subfamily E member 1 (OR51E1), olfactory receptor family 1 subfamily A member 1 (OR1A1), olfactory receptor family 2 subfamily C member 1 (OR2C1), and olfactory receptor family 10 subfamily J member 5 (OR10J5) (referred to herein as “GLP-1/PYY secretion compositions”).

In a preferred embodiment, the GLP-1/PYY secretion compositions of the invention, when delivered to a patient or subject, thus, effectuate the following highly effective and, hence, desirable pharmacodynamic activity.

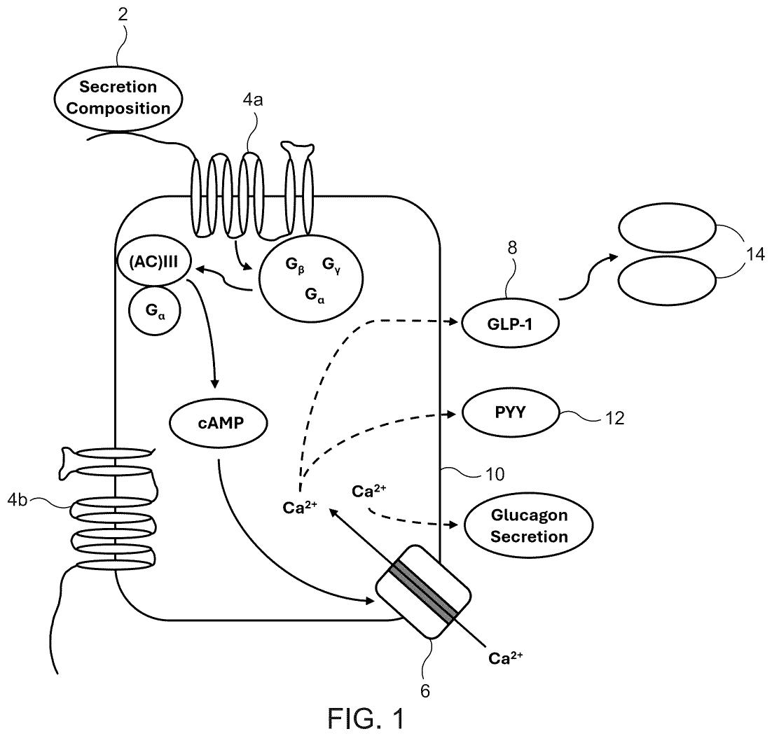

Referring to FIG. 1, in a preferred embodiment, the GLP-1/PYY secretion compositions (denoted “2”) of the invention target and bind to ectopic olfactory receptors (ORs) (denoted “4a, 4b”) disposed on enteroendocrine cells (in this instance, enteroendocrine L-cells) and pancreatic α-cells (both denoted “10”). The glucose-induced membrane depolarization of the enteroendocrine and α-cells 10 opens the voltage-dependent Ca²⁺ (VDC) channels (denoted “6”) of the enteroendocrine and α-cells 10, and the resulting Ca²⁺ influx triggers vesicular exocytosis and increases secretion of GLP-1 (denoted “8”) and PYY (denoted “12”) from the cells.

The secreted GLP-1 binds to and activates GLP-1 receptor proteins on pancreatic β-cells (denoted “14”), which, as indicated above, induces secretion of insulin.

The secreted insulin also binds to insulin receptors (IR) of the endogenous hepatic cells to suppress hepatic glucose output by inhibiting adipose lipolysis and, thereby, release of glucose into an individual's bloodstream.

The secreted GLP-1 also binds to and activates GLP-1 receptor proteins on endogenous GI cells, such as islet cells of the pancreas, whereby glucagon release is suppressed. The secreted GLP-1 also suppresses glucagon secretion indirectly via its insulinotropic effect on the pancreatic β-cells, more particularly, the secretion of further suppressors of glucagon secretion by endogenous GI cells, e.g., amylin, zinc, and γ-aminobutyric acid (GABA).

The secreted GLP-1 can also bind to GLP-1 receptor proteins on endogenous brain cells, more specifically, glutamatergic neurons of the hindbrain, which can, and often will, decrease the rate of gastric emptying and acid secretion, and, thereby, reduce appetite.

The binding of GLP-1 to the GLP-1 receptor proteins on endogenous enteroendocrine cells also induces peptide Y-Y (PYY) secretion by the endogenous enteroendocrine cells. PYY then subsequently binds to neuropeptide Y receptors on local central nervous system cells, which activate seminal cell signaling cascades that increase the efficiency of digestion and nutrient absorption after meal consumption to promote satiety and suppress appetite.

The GLP-1/PYY secretion compositions of the invention (and GLP-1/GIP secretion compositions of the invention, discussed below), when delivered to a patient or subject, thus induce GLP-1 and/or PYY secretion in vivo, whereby insulin secretion of the patient is induced, and the appetite of the patient is suppressed.

The GLP-1/PYY secretion compositions of the invention (and GLP-1/GIP secretion compositions of the invention) thus provide an effective means of treating diabetes mellitus, and particularly type-2 diabetes mellitus, and obesity.

According to the invention, the GLP-1/PYY secretion compositions of the invention can comprise at least one of the receptor activating compounds listed below in Table I below.

TABLE I

| | | | Signaling | Biological |
|---|---|---|---|---|
| Receptor | Expression Site | Compounds/Ligands | Pathway | Process |
| OR51E1 | Enteroendocrine | nonanoic acid | AC3-cAMP | Increased |
| | L-Cell | butyl butyryl lactate | | GLP-1 |
| | | farnesol | | Secretion and |
| | | 3-methylpentanoic acid | | Activation |
| | | 4-methylpentanoic acid | | |
| | | eugenol | | |
| | | isovaleric acid | | |
| | | nonanoic acid | cAMP- | Increased |
| | | pentanol | Mediated | Peptide Y-Y |
| | | farnesol | | Secretion and |
| | | 3-methylpentanoic acid | | Activation |
| | | 4-methylpentanoic acid | | |
| | | eugenol | | |
| | | isovaleric acid | | |
| OR1A1 | Enteroendocrine | citronellal | AC3-cAMP | |
| | L-Cell | hydroxycitronellal | | |
| | | citral | | |
| | | geraniol | | |
| | | 3-methyl-2,4- | cAMP- | Increased GLP-1 |
| | | nonanedione | Mediated | Secretion and |
| | | estragole | | Activation |
| | | neroli | | |
| | | heptanol | | |
| | | octanol | | |
| | | helional | | |
| | | nonanal | | |
| OR2C1 | Pancreatic | octanoic acid | PLC-IP₃ | Upregulation of |
| | β-Cells | | | Glycerol Kinase |
| | | | | Protein |
| | | eugenol | cAMP- | Upregulation of |
| | | musk ketone | Mediated | Glycerol Kinase |
| | | (+)-dihydrocarvone | | Protein |
| OR10J5 | Hepatocytes | α-cedrene | cAMP-PKA- | Downregulation |
| | | lyral | CREB | of Lipogenesis |
| | | thujopsene | cAMP-PKA- | Genes |
| | | | AMPK | |

As indicated above, the GLP-1/PYY secretion compositions of the invention can also comprise any combination of the aforementioned receptor activating compounds.

In a preferred embodiment, the GLP-1/PYY secretion compositions of the invention (and GLP-1/GIP secretion compositions of the invention, discussed below) are specifically adapted to modulate activity of at least OR51E1 when the GLP-1/PYY secretion compositions of the invention are delivered to a patient or subject.

Modulation of OR51E1 and the desirable pharmacodynamic activity resulting therefrom is described below.

### OR51E1 Modulation

As indicated above, in a preferred embodiment of the invention, the GLP-1/PYY secretion compositions of the invention comprise at least one receptor activating compound and/or ligand that is adapted to modulate the activity of OR51E1 (referred to herein as “OR51E1 activating compounds”).

According to the invention, activation of OR51E1 by an OR51E1 activating compound induces a glucose-induced membrane depolarization of endogenous GI cells, more particularly, L-enteroendocrine and pancreatic α-cells and, thereby, opens the voltage-dependent Ca²⁺ (VDC) channels of the L- and α-cells, and the resulting Ca²⁺ influx triggers vesicular exocytosis and increases secretion of GLP-1.

The secreted GLP-1 binds to and activates GLP-1 receptor proteins on pancreatic β-cells, which, as indicated above, induces secretion of insulin into a subject's blood stream.

The secreted insulin then binds to the insulin receptors (IR) of endogenous cells to effectuate the activation of cell signaling cascades that modulate energy metabolism and decrease blood glucose.

In a preferred embodiment, the activation of OR51E1 effectuates secretion modulation and/or activation of at least one of the aforementioned additional endocrine factors.

According to the invention, the activation of OR51E1 can also effectuate secretion modulation and/or activation of additional endocrine factors, including, without limitation, ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, glucagon, enteroglucagon, galanin, leptin, motilin, amylin, neuropeptide Y (NPY), pancreatic polypeptide, substance P, oxyntomodulin, and somatostatin.

In some embodiments, the GLP-1/PYY secretion compositions of the invention comprise a single OR51E1 activating compound, such as butyl butyryl lactate, which is adapted to bind to and activate OR51E1.

In some embodiments, the GLP-1/PYY secretion compositions of the invention comprise a combination of OR51E1 activating compounds, such as eugenol and butyl butyryl lactate, which are similarly preferably adapted to bind to and activate OR51E1.

According to the invention, the GLP-1/PYY secretion compositions of the invention can comprise any suitable combination of the OR51E1 activating compounds referenced above.

In some embodiments of the invention, the GLP-1/PYY secretion compositions of the invention (and, as discussed below, GLP-1/GIP compositions of the invention) comprise at least one of the following OR51E1 activating compounds: eugenol, butyl butyryl lactate, 3-methylpentanoic acid, farnesol, isovaleric acid, 4-methylpentanoic acid, and nonanoic acid.

In a preferred embodiment, the GLP-1/PYY secretion compositions of the invention (and, as discussed below, GLP-1/GIP compositions of the invention) comprise at least butyl butyryl lactate, which, as reflected in the Examples below, induces unexpected levels of OR51E1 activity.

As indicated above, in some embodiments, the GLP-1/PYY secretion compositions of the invention (and GLP-1/GIP secretion compositions of the invention, discussed below) are formed in tablet or capsule form to accommodate oral (or enteric) and sublingual delivery to a subject.

According to the invention, the tablets and capsules comprising the GLP-1/PYY secretion compositions (and GLP-1/GIP secretion compositions) of the invention can comprise any ex vivo mass in the range of approximately 100.0 mg to approximately 10000.0 mg.

In a preferred embodiment, the ex vivo mass of a GLP-1/PYY secretion composition (and GLP-1/GIP secretion composition) of the invention is in the range of approximately 500.0 mg to approximately 2500.0 mg.

In some embodiments, the ex vivo mass of a GLP-1/PYY secretion composition (and GLP-1/GIP secretion composition) of the invention is in the range of approximately 1200.0 mg to approximately 1400.0 mg. In some embodiments, the ex vivo mass of a tablet or capsule is in the range of approximately 600.0 mg to approximately 800.0 mg.

In a preferred embodiment, the ex vivo mass of an OR51E1 activating compound contained in a GLP-1/PYY secretion composition (and GLP-1/GIP secretion composition) of the invention comprises at least approximately 1.0 μg.

According to the invention, the ex vivo mass of an OR51E1 activating compound contained in a GLP-1/PYY secretion composition (and GLP-1/GIP secretion composition) of the invention can thus comprise at least approximately 10.0 μg, approximately 15.0 μg, approximately 20.0 μg, approximately 25.0 μg, approximately 30.0 μg, approximately 35.0 μg, approximately 40.0 μg, approximately 45.0 μg, approximately 50.0 μg, approximately 55.0 μg, approximately 60.0 μg, approximately 65.0 μg, approximately 70.0 μg, approximately 75.0 μg, approximately 80.0 μg, approximately 85.0 μg, approximately 90.0 μg, approximately 95.0 μg, approximately 100.0 μg, approximately 1.0 mg, approximately 2.0 mg, approximately 3.0 mg, approximately 4.0 mg, approximately 5.0 mg, approximately 6.0 mg, approximately 7.0 mg, approximately 8.0 mg, approximately 9.0 mg, approximately 10.0 mg, approximately 20.0 mg, approximately 30.0 mg, approximately 40.0 mg, approximately 50.0 mg, approximately 60.0 mg, approximately 70.0 mg, approximately 80.0 mg, approximately 90.0 mg, approximately 100.0 mg, approximately 150.0 mg, approximately 200.0 mg, approximately 250.0 mg, approximately 300.0 mg, approximately 350.0 mg, approximately 400.0 mg, approximately 450.0 mg, approximately 500.0 mg, approximately 600.0 mg, approximately 700.0 mg, approximately 800.0 mg, approximately 900.0 mg, or approximately 1.0 g.

According to the invention, the ex vivo mass of the noted OR51E1 activating compounds contained in a GLP-1/PYY secretion composition (and GLP-1/GIP secretion composition) of the invention can also comprise in the range of approximately 1.0 μg to approximately 10.0 g.

Thus, according to the invention, the ex vivo mass of an OR51E1 activating compound contained in a GLP-1/PYY secretion composition (and GLP-1/GIP secretion composition) of the invention can also comprise in the range of approximately 10.0 μg to approximately 50.0 μg, approximately 10.0 μg to approximately 100.0 μg, approximately 10.0 μg to approximately 200.0 μg, approximately 200.0 μg to approximately 400.0 μg, approximately 300.0 μg to approximately 600.0 μg, approximately 400.0 μg to approximately 800.0 μg, approximately 500.0 μg to approximately 1.0 mg, approximately 1.0 mg to approximately 2.0 mg, approximately 1.0 mg to approximately 5.0 mg, approximately 1.0 mg to approximately 10.0 mg, approximately 5.0 mg to approximately 10.0 mg, approximately 5.0 mg to approximately 20.0 mg, approximately 5.0 mg to approximately 30.0 mg, approximately 10.0 mg to approximately 20.0 mg, approximately 10.0 mg to approximately 40.0 mg, approximately 10.0 mg to approximately 50.0 mg, approximately 20.0 mg to approximately 50.0 mg, approximately 20.0 mg to approximately 40.0 mg, approximately 20.0 mg to approximately 60.0 mg, approximately 30.0 mg to approximately 50.0 mg, approximately 30.0 mg to approximately 70.0 mg, approximately 40.0 mg to approximately 80.0 mg, approximately 40.0 mg to approximately 100.0 mg, approximately 100.0 mg to approximately 200.0 mg, approximately 100.0 mg to approximately 300.0 mg, approximately 200.0 mg to approximately 300.0 mg, approximately 200.0 mg to approximately 400.0 mg, approximately 200 mg to approximately 600.0 mg, approximately 100.0 mg to approximately 1.0 g, and approximately 1.0 g to approximately 10.0 g, and/or any ex vivo mass therebetween.

According to the invention, an OR51E1 activating compound contained in a GLP-1/PYY secretion composition (and GLP-1/GIP secretion composition) of the invention can also comprise an EC₅₀ concentration range in the range of approximately 1.0 nM to approximately 1000.0 mM.

In a preferred embodiment, the EC₅₀ concentration of an OR51E1 activating compound contained in a GLP-1/PYY secretion composition (and GLP-1/GIP secretion composition) of the invention comprises at least 0.001 μM.

According to the invention, the EC₅₀ concentration of an OR51E1 activating compound contained in a GLP-1/PYY secretion composition (and GLP-1/GIP secretion composition) of the invention can thus comprise at least 0.002 μM, at least 0.003 μM, at least 0.004 μM, at least 0.005 μM, at least 0.006 μM, at least 0.007 μM, at least 0.008 μM, at least 0.009 μM, at least 0.01 μM, at least 0.02 μM, at least 0.03 μM, at least 0.04 μM, at least 0.05 μM, at least 0.06 μM, at least 0.07 μM, at least 0.08 μM, at least 0.09 μM, at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, at least 1.0 μM, at least 2.0 μM, at least 3.0 μM, at least 4.0 μM, at least 5.0 μM, at least 6.0 μM, at least 7.0 μM, at least 8.0 μM, at least 9.0 μM, at least 10.0 μM, at least 20.0 μM, at least 30.0 μM, at least 40.0 μM, at least 50.0 μM, at least 60.0 μM, at least 70.0 μM, at least 80.0 μM, at least 90.0 μM, at least 100.0 μM, at least 200.0 μM, at least 300.0 μM, at least 400.0 μM, at least 500.0 μM, at least 600.0 μM, at least 700.0 μM, at least 800.0 μM, at least 900.0 μM, or at least 1,000.0 μM.

As indicated above, an OR51E1 activating compound contained in a GLP-1/PYY secretion composition of the invention can also comprise an EC₅₀ concentration range in the range of approximately 1.0 nM to approximately 1000.0 mM.

Thus, according to the invention, the EC₅₀ concentration of an OR51E1 activating compound contained in a GLP-1/PYY secretion composition (and GLP-1/GIP secretion composition) of the invention can also comprise in the range of approximately 0.001 μM to approximately 100000.0 μM, approximately 0.002 μM to approximately 10000.0 μM, approximately 0.003 μM to approximately 1000.0 μM, approximately 0.005 μM to approximately 750.0 μM, approximately 0.01 μM to approximately 500.0 μM, approximately 0.05 μM to approximately 50.0 μM, approximately 0.05 μM to approximately 100.0 μM, approximately 0.05 μM to approximately 150.0 μM, approximately 0.05 μM to approximately 200.0 μM, approximately 0.05 μM to approximately 250.0 μM, approximately 0.05 μM to approximately 300.0 μM, approximately 0.05 μM to approximately 350.0 μM, approximately 0.05 μM to approximately 400.0 μM, approximately 0.05 μM to approximately 450.0 μM, approximately 0.05 μM to approximately 500.0 μM, approximately 0.1 μM to approximately 50.0 μM, approximately 0.1 μM to approximately 100.0 μM, approximately 0.1 μM to approximately 150.0 μM, approximately 0.1 μM to approximately 200.0 μM, approximately 0.1 μM to approximately 250.0 μM, approximately 0.1 μM to approximately 300.0 μM, approximately 0.1 μM to approximately 350.0 μM, approximately 0.1 μM to approximately 400.0 μM, approximately 0.1 μM to approximately 450.0 M, approximately 0.1 μM to approximately 500.0 μM, approximately 0.1 μM to approximately 1000.0 μM, approximately 0.1 μM to approximately 1500.0 μM, approximately 0.1 μM to approximately 2000.0 μM, approximately 0.1 μM to approximately 2500.0 μM, approximately 0.1 μM to approximately 3000.0 μM, approximately 0.25 μM to approximately 50.0 μM, approximately 0.25 μM to approximately 100.0 μM, approximately 0.25 μM to approximately 150.0 μM, approximately 0.25 μM to approximately 200.0 μM, approximately 0.25 μM to approximately 250.0 μM, approximately 0.25 μM to approximately 300.0 μM, approximately 0.25 μM to approximately 350.0 μM, approximately 0.25 μM to approximately 400.0 μM, approximately 0.25 μM to approximately 450.0 μM, approximately 0.25 μM to approximately 500.0 μM, approximately 0.5 μM to approximately 300.0 μM, approximately 1.0 μM to approximately 50.0 μM, approximately 1.0 μM to approximately 100.0 M, approximately 1.0 μM to approximately 150.0 μM, approximately 1.0 μM to approximately 200.0 μM, approximately 1.0 μM to approximately 250.0 μM, approximately 1.0 μM to approximately 300.0 μM, approximately 1.0 μM to approximately 350.0 μM, approximately 1.0 μM to approximately 400.0 μM, approximately 1.0 μM to approximately 450.0 μM, approximately 1.0 μM to approximately 500.0 μM, approximately 2.5 μM to approximately 100.0 μM, approximately 5.0 M to approximately 75.0 μM, approximately 7.5 M to approximately 50.0 M, approximately 10.0 μM to approximately 25.0 μM, and/or any EC₅₀ concentrations therebetween.

According to the invention, the EC₅₀ concentration of an OR51E1 activating compound contained in a GLP-1/PYY secretion composition (and GLP-1/GIP secretion composition) of the invention can also comprise in the range of approximately 0.001 μM to approximately 10.0 μM, approximately 0.005 μM to approximately 7.5 μM, approximately 0.01 μM to approximately 5.0 μM, approximately 0.03 μM to approximately 2.5 μM, approximately 0.05 μM to approximately 1.5 μM, approximately 0.03 μM to approximately 1.0 μM, approximately 0.1 μM to approximately 0.5 μM, and/or any EC₅₀ concentrations therebetween.

As indicated above, in some embodiments of the invention, the GLP-1/PYY secretion compositions (and GLP-1/GIP secretion compositions) of the invention comprises at least one of the following OR51E1 activating compounds: eugenol, butyl butyryl lactate, 3-methylpentanoic acid, farnesol, isovaleric acid, 4-methylpentanoic acid, and nonanoic acid.

As also indicated above, in a preferred embodiment, the GLP-1/PYY secretion compositions (and GLP-1/GIP secretion compositions) comprise at least butyl butyryl lactate, which, as indicated above and reflected in the Examples below, induces unexpected levels of OR51E1 activity.

According to the invention, the mass of the noted OR51E1 activating compounds in a GLP-1/PYY secretion composition (and GLP-1/GIP secretion composition) of the invention can comprise any of the aforementioned ex vivo masses and mass ranges.

According to the invention, the noted OR51E1 activating compounds can also comprise any of the aforementioned EC₅₀ concentrations and EC₅₀ concentration ranges in a GLP-1/PYY secretion composition (and GLP-1/GIP secretion composition) of the invention.

### OR1A1 Modulation

In some embodiments, the GLP-1/PYY secretion compositions (and GLP-1/GIP secretion compositions) of the invention also comprise one or more receptor activating compounds and/or ligands that are specifically adapted to bind to and activate OR1A1 (referred to herein as “OR1A1 activating compounds”), whereby pharmacodynamic activity similar to that induced via activation of OR51E1 (discussed above) is induced.

According to the invention, the OR1A1 activating compounds can comprise, without limitation, geraniol, citronellol, 3-methyl-2,4-nonanedione, estragole, neroli, heptanol, octanol, helional, nonanal, hydroxycitronellal, and citral.

According to the invention, the mass of the noted OR1A1 activating compounds in a GLP-1/PYY secretion composition (and GLP-1/GIP secretion composition) of the invention can similarly comprise any of the aforementioned ex vivo masses and mass ranges.

According to the invention, the noted OR1A1 activating compounds can also comprise any of the aforementioned EC₅₀ concentrations and EC₅₀ concentration ranges in a GLP-1/PYY secretion composition (and GLP-1/GIP secretion composition) of the invention.

### OR2C1 Modulation

In some embodiments, the GLP-1/PYY secretion compositions (and GLP-1/GIP secretion compositions) of the invention also comprise one or more receptor activating compounds and/or ligands that are specifically adapted to activate OR2C1 (referred to herein as “OR2C1 activating compounds”), whereby the following pharmacodynamic activity is induced.

Activation of OR2C1 induces Ca²⁺ release from the endoplasmic reticulum of pancreatic β-cells through the phospholipase C-inositol triphosphate-dependent (PLC-IP3) pathway and, thereby, an increased concentration of intracellular Ca²⁺. The increase in intracellular Ca²⁺ then activates the CaMKK/CaMKIV pathway, which induces glucokinase (GK) expression, thereby inducing glucose absorption by endogenous cells and glucose-stimulated insulin secretion (GSIS) from pancreatic islet cells.

The secreted insulin then similarly binds to the insulin receptors (IR) of endogenous cells to effectuate the activation of cell signaling cascades that modulate energy metabolism and decrease blood glucose.

In a preferred embodiment, the activation of OR2C1 similarly effectuates secretion modulation and/or activation of at least one of the aforementioned additional endocrine factors.

According to the invention, the GLP-1/PYY secretion compositions of the invention (and GLP-1/GIP secretion compositions of the invention) can comprise one or more of the following OR2C1 activating compounds: eugenol, octanoic acid, musk ketone, and (+)-dihydrocarvone.

According to the invention, the mass of the noted OR2C1 activating compounds in a GLP-1/PYY secretion composition (and GLP-1/GIP secretion composition) of the invention can similarly comprise any of the aforementioned ex vivo masses and mass ranges.

According to the invention, the noted OR2C1 activating compounds can also comprise any of the aforementioned EC₅₀ concentrations and EC₅₀ concentration ranges in a GLP-1/PYY secretion composition (and GLP-1/GIP secretion composition) of the invention.

### OR10J5 Modulation

In some embodiments of the invention, the GLP-1/PYY secretion compositions (and GLP-1/GIP secretion compositions) of the invention also comprise one or more receptor activating compounds and/or ligands that are specifically adapted to activate OR10J5 (referred to herein as “OR10J5 activating compounds”), whereby the following pharmacodynamic activity is induced.

Activation of OR10J5 induces downregulation of the seminal lipogenesis associated gene expression, including the expression of C/EBPα, PPARγ, RXR, LXRα, SREBP-1c, ap2, FAS, SCD1, ACC, and mtGPAT genes, and upregulation of mitochondrial and thermogenic gene expression, including the expression of PGC-1α, PRDM16, UCP1, Cyte, Cox4, and Cidea genes through the cAMP/PKA/HSL pathway.

The above noted downregulation of the seminal lipogenesis associated gene expression and upregulation of mitochondrial and thermogenic gene expression modulates lipid metabolism by inhibiting lipogenesis and, thus, reducing lipid accumulation in hepatic cells.

In a preferred embodiment, the activation of OR10J5 similarly effectuates secretion modulation and/or activation of at least one of the aforementioned additional endocrine factors.

According to the invention, the GLP-1/PYY secretion compositions of the invention (and GLP-1/GIP secretion compositions of the invention) can comprise one or more of the following OR10J5 activating compounds: a-cedrene, hydroxymethyl-pentylcyclohexenecarboxaldehyde (also referred to as “lyral”), and thujopsene.

According to the invention, the mass of the noted OR10J5 activating compounds in a GLP-1/PYY secretion composition (and GLP-1/GIP secretion composition) of the invention can similarly comprise any of the aforementioned ex vivo masses and mass ranges.

According to the invention, the noted OR10J5 activating compounds can also comprise any of the aforementioned EC₅₀ concentrations and EC₅₀ concentration ranges in a GLP-1/PYY secretion composition (and GLP-1/GIP secretion composition) of the invention.

### Olfactory Receptor-Mediated and Free Fatty Acid Receptor-Mediated (GIP) Secretion

In some embodiments of the invention, the compositions of the invention comprise one or more receptor activating compounds and/or ligands that are adapted to bind to and activate at least one receptor that induces GIP secretion in vivo including, without limitation, free fatty acid receptor 1 (FFAR1), free fatty acid receptor 4 (FFAR4), olfactory receptor family 2, subfamily W, member 1 (OR2W1), olfactory receptor family 2, subfamily B, member 11 (OR2B11), olfactory receptor family 2 subfamily J member 3 (OR2J3), and transient receptor potential cation channel, subfamily A, member 1 (TRPA1) (referred to herein as “GIP secretion compositions”).

According to the invention, the GIP secretion compositions (and GLP-1/GIP secretion compositions) of the invention, when delivered to a patient or subject, effectuate the following highly effective and, hence, desirable pharmacodynamic activity.

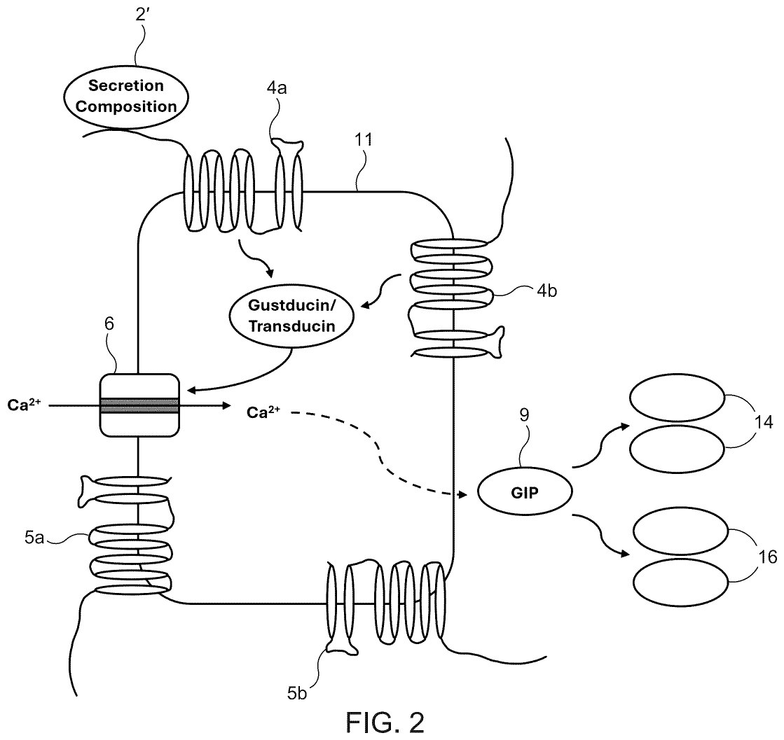

Referring now to FIG. 2, in a preferred embodiment, when a GIP secretion composition of the invention (and GLP-1/GIP secretion composition of the invention) (denoted “2”) targets and binds to ectopic olfactory receptors (denoted “4a, 4b”) and free fatty acid receptors (denoted “5a, 5b”) disposed on endogenous cells (in this instance, enteroendocrine cells) (denoted “11”), a gustducin-mediated cell signaling pathway is activated, which induces membrane depolarization of the enteroendocrine cells 11 and opens the voltage-dependent Ca²⁺ (VDC) channels (denoted “6”) of the enteroendocrine cells 11, wherein the resulting Ca²⁺ influx induces vesicular exocytosis and increased secretion (denoted “9”) of GIP from the enteroendocrine cells 11.

The secreted GIP binds to and activates GIP receptor proteins on pancreatic β-cells (denoted “14”), which, induces secretion of insulin.

The secreted insulin then binds to the insulin receptors (IRs) of endogenous cells to effectuate the activation of cell signaling cascades that modulate energy metabolism and decrease blood glucose.

The secreted insulin also binds to the IRs of the endogenous hepatic cells to suppress hepatic glucose output by inhibiting adipose lipolysis and, thereby, release of glucose into an individual's bloodstream.

The secreted GIP also binds to and activates GIP receptor proteins on endogenous GI cells (denoted “16”), such as islet cells of the pancreas, to promote pancreatic β-cell survival and prevent apoptosis of pancreatic β-cells by activating the cAMP response element-binding (CREB) and Akt/PKB pathways, thus, directly, and indirectly maintaining a stable population of insulin-producing pancreatic β-cells in vivo.

The GIP secretion compositions of the invention (and GLP-1/GIP secretion compositions of the invention), when delivered to a patient or subject, thus similarly induce GIP secretion in vivo, whereby insulin secretion of the patient is induced and the appetite of the patient is suppressed.

The GIP secretion compositions of the invention (and GLP-1/GIP secretion compositions of the invention) thus provide an effective means of treating diabetes mellitus, and particularly type-2 diabetes mellitus, and obesity.

According to the invention, the GIP secretion compositions (and GLP-1/GIP secretion compositions) of the invention can comprise at least one of the receptor activating compounds/ligands listed below in Table II below.

TABLE II

| | | | Signaling | Biological |
|---|---|---|---|---|
| Receptor | Expression Site | Compounds/Ligands | Pathway | Process |
| FFAR1 | Enteroendocrine | medium-chain free | Gustducin/ | Increased GIP |
| | L-Cells | fatty acids (e.g., lauric acid | Transducin | Secretion and |
| | | (c12:0) | | Activation |
| FFAR4 | Enteroendocrine | long-chain free fatty | | |
| | L-Cells | acids (e.g., stearic acid) | | |
| | Pancreatic | omega-3 | | Inhibition of |
| | δ-Cells | polyunsaturated fatty | | Somatostatin |
| | | acids (e.g., alpha- | | to Promote |
| | | linoleic acid, | | Insulin |
| | | docosahexaenoic | | Secretion |
| | | acid, and | | |
| | | eicosatetraenoic acid) | | |
| OR2W1 | Enteroendocrine | 2-heptanone | AC3-cAMP | Increased GIP |
| | L-Cells | 1-octanal | | Secretion and |
| | Adipose Tissue | (−)-citronellol | | Activation |
| | Cells | Hexanal | | |
| | | 3-octanone | | |
| | | hexyl acetate | | |
| | | 1-hexanol | | |
| | | octanoic acid | | |
| | | 1-heptanol | | |
| | | allyl phenylacetate | | |
| | | benzyl acetate | | |
| | | 3,4-hexanedione | | |
| | | cis-3-hexen-1-ol | | |
| OR2B11 | Enteroendocrine | 2-ethyl-3,5- | AC3-cAMP | Increased GIP |
| | L-Cells | dimethylpyrazine | | Secretion and |
| | | coumarin | | Activation |
| | | dicyclohexyl disulfide | | |
| | | spearmint | | |
| | | coffee difuran | | |
| | | quinoline | | |
| | | cinnamaldehyde | | |
| OR2J3 | Enteroendocrine | cis-3-hexen-1-ol | AC3-cAMP | Increased GIP |
| | L-Cells | cinnamaldehyde | | Secretion and |
| | | | | Activation |
| TRPA1 | Enterochromaffin | allyl isothiocyanate | Ca²⁺ | Increased GIP |
| | Cells | cinnamaldehyde | | Secretion and |
| | | farnesyl thiosalicylic | | Activation |
| | | acid | | |
| | | formalin | | |
| | | hydrogen peroxide | | |
| | | 4-hydroxynonenal | | |
| | | acrolein | | |

As indicated above, the GIP secretion compositions (and GLP-1/GIP secretion compositions) of the invention can also comprise any combination of the aforementioned receptor activating compounds.

In some embodiments of the invention, the GIP secretion compositions (and GLP-1/GIP secretion compositions) of the invention comprise at least one of the receptor activating compounds and/or ligands that are set forth in Table II above, which are specifically adapted to activate at least FFAR1 and/or FFAR4 in vivo (referred to herein as “FFAR activating compounds”).

In a preferred embodiment, the GIP secretion compositions (and GLP-1/GIP secretion compositions) of the invention comprise at least one FFAR activating compound that is specifically adapted to activate at least FFAR1 in vivo.

Modulation of FFAR1 and FFAR4 and the desirable pharmacodynamic activity resulting therefrom is described below.

### FFAR1 and FFAR4 Modulation

As is well established by published studies^{1, 2}, activation of FFAR1 and FFAR4 by the FFAR activating compounds of the invention induces activation of a gustducin-mediated cell signaling pathway, which induces membrane depolarization of enteroendocrine cells and opens voltage-dependent Ca²⁺ (VDC) channels of the enteroendocrine cells, wherein the resulting Ca²⁺ influx induces increased secretion of GIP from the enteroendocrine cells.

The activation of FFAR1 and/or FFAR4, can, in some instances, also induce activation of G_{aq/11} and β-arrestin signaling pathways and, thereby, stimulate further GIP secretion.

The secreted GIP binds to and activates GIP receptor proteins on pancreatic β-cells, which induces secretion of insulin.

The secreted insulin then binds to the insulin receptors (IR) of endogenous cells to effectuate the activation of cell signaling cascades that modulate energy metabolism and decrease blood glucose.

The secreted insulin also binds to insulin receptors (IR) of the endogenous hepatic cells to suppress hepatic glucose output by inhibiting adipose lipolysis and, thereby, release of glucose into an individual's bloodstream.

The secreted GIP also binds to and activates GIP receptor proteins on endogenous GI cells, such as islet cells of the pancreas, to promote pancreatic β-cell survival and prevent apoptosis of pancreatic β-cells by activating the CAMP response element-binding (CREB) and Akt/PKB pathways, thus, directly and indirectly maintaining a stable population of insulin-producing pancreatic β-cells in vivo.

In a preferred embodiment, activation of FFAR1 and/or FFAR4, also effectuates secretion modulation and/or activation of at least one of the aforementioned additional endocrine factors.

According to the invention, the FFAR activating compounds can comprise a medium-chain free fatty acid, a long-chain free fatty acid, and an omega-3 polyunsaturated fatty acid.

In a preferred embodiment, the FFAR activating compounds comprise a medium-chain free fatty acid.

The pharmacodynamic activity induced via activation of FFAR1 and FFAR4 by a medium-chain free fatty acid is summarized below.

As is well established, a medium-chain free fatty acid is a fatty acid comprising a carboxylic acid head group with a 6-12 carbon aliphatic chain (saturated or unsaturated) with a methyl group on the end of the aliphatic chain. Foods that are rich in medium chain fatty acids include, without limitation, palm kernel oil and coconut oil.

According to the invention, when a medium-chain free fatty acid binds to a free fatty acid receptor (FFAR), such as FFAR1 or FFAR4, the medium-chain free fatty acid induces a conformational change in the molecular structure of the FFAR that induces the intracellular G_{α}/G_{β}/G_{γ} subunits of the FFAR to act as a guanine nucleotide exchange factor and, thus, exchange a guanine diphosphate (GDP) for a guanine triphosphate (GTP), which binds to the Ga subunit of the FFAR1 and FFAR4.

The noted binding of the GTP to the Ga subunit then induces a dissociation of the G_{α}/G_{β}/G_{γ} subunits of the FFAR1 and FFAR4 into a (i) free Ga subunit and a (ii) G_{β}/G_{γ} complex and, thereby, activates seminal downstream cell signaling processes that induce an increase in intracellular cAMP in endogenous cells, such as enteroendocrine cells.

As depicted in FIG. 2, by virtue of the intracellular CAMP level increase, the opening of cyclic nucleotide gated Ca²⁺ channels is induced, which results in increased cellular Ca²⁺ and, thereby, induction of increased secretion of GIP from the endogenous cells.

The secreted GIP binds to and activates GIP receptor proteins on pancreatic β-cells, which, as indicated above, (i) induces secretion of insulin, (ii) promotes pancreatic β-cell survival, and (iii) prevents apoptosis of pancreatic β-cells by activating the CAMP response element-binding (CREB) and Akt/PKB pathways, thus, directly, and indirectly maintaining a stable population of insulin-producing pancreatic β-cells in vivo.

In some embodiments of the invention, the medium-chain free fatty acid comprises at least one of the following FFAR activating compounds: lauric acid and capric acid.

In a preferred embodiment, the medium-chain fatty acid and, hence, FFAR activating compound comprises lauric acid, which, as reflected in the Examples below, is adapted to bind to and induce optimal FFAR1 activity.

According to the invention, the mass of the noted FFAR activating compounds in a GIP secretion composition (and GLP-1/GIP secretion composition) of the invention can similarly comprise any of the aforementioned ex vivo masses and mass ranges.

According to the invention, the noted FFAR activating compounds can also comprise any of the aforementioned EC₅₀ concentrations and EC₅₀ concentration ranges in a GIP secretion composition (and GLP-1/GIP secretion composition) of the invention.

As indicated above, according to the invention, the GIP secretion compositions (and GLP-1/GIP secretion compositions) of the invention can also comprise one or more receptor activating compounds and/or ligands that are specifically adapted to modulate the activity of olfactory receptor family 2, subfamily W, member 1 (OR2W1), olfactory receptor family 2 subfamily B member 11 (OR2B11), olfactory receptor family 2 subfamily J member 3 (OR2J3) and transient receptor potential cation channel subfamily A member 1 (TRPA1), whereby GIP secretion in vivo is similarly induced.

Modulation of OR2W1, OR2B11, OR2J3 and TRPA1, and the desirable pharmacodynamic activity resulting therefrom is described below.

### OR2W1 Modulation

In some embodiments of the invention, the GIP secretion compositions of the invention (and GLP-1/GIP secretion compositions of the invention) thus comprise one or more receptor activating compounds and/or ligands that are adapted to modulate the activity of OR2W1, more particularly, activate OR2W1 (referred to herein as “OR2W1 activating compounds”), whereby, as discussed below, a conformational change is induced in the molecular structure of OR2W1.

According to the invention, when a OR2W1 activating compound, e.g., benzyl acetate, binds to OR2W1, the OR2W1 activating compound induces a conformational change in the molecular structure of the OR2W1 that similarly induces the intracellular G_{α}/G_{β}/G_{γ} subunits of the olfactory receptor to act as a guanine nucleotide exchange factor and, thus, exchange a guanine diphosphate (GDP) for a guanine triphosphate (GTP), which binds to the Ga subunit of the OR2W1.

The noted binding of the GTP to the Ga subunit then induces a dissociation of the G_{α}/G_{β}/G_{γ} subunits of the OR2W1 into a (i) free Ga subunit and a (ii) G_{β}/G_{γ} complex and, thereby, activates seminal downstream cell signaling processes that induce an increase in intracellular CAMP in endogenous cells, such as enteroendocrine cells.

As depicted in FIG. 2, by virtue of the intracellular cAMP level increase, the opening of cyclic nucleotide gated Ca²⁺ channels is induced, which results in increased cellular Ca²⁺ and, thereby, increased induction of secretion of GIP from the endogenous cells.

The secreted similarly GIP binds to and activates GIP receptor proteins on pancreatic β-cells, which, as indicated above, (i) induces secretion of insulin, (ii) promotes pancreatic β-cell survival, and (iii) prevents apoptosis of pancreatic β-cells by activating the CAMP response element-binding (CREB) and Akt/PKB pathways, thus, directly and indirectly maintaining a stable population of insulin-producing pancreatic β-cells in vivo.

In some embodiments, the activation of OR2W1 similarly effectuates secretion modulation and/or activation of at least one of the aforementioned additional endocrine factors.

According to the invention, the GIP secretion compositions (and GLP-1/GIP secretion compositions) of the invention can comprise one or more of the following OR2W1 activating compounds: benzyl acetate, 2-heptanone, 1-octanal, (−)-citronellol, hexanal, 3-octanone, hexyl acetate, 1-hexanol, octanoic acid, 1-heptanol, allyl phenylacetate, 3,4-hexanedione, and cis-3-hexen-1-ol.

In a preferred embodiment, the OR2W1 activating compound comprises benzyl acetate.

According to the invention, the mass of the noted OR2W1 activating compounds in a GIP secretion composition (and GLP-1/GIP secretion composition) of the invention can similarly comprise any of the aforementioned ex vivo masses and mass ranges.

According to the invention, the noted OR2W1 activating compounds can also comprise any of the aforementioned EC₅₀ concentrations and EC₅₀ concentration ranges in a GLP-1/PYY secretion composition (and GLP-1/GIP secretion composition) of the invention.

### OR2B11 Modulation

In some embodiments of the invention, the GIP secretion compositions of the invention (and GLP-1/GIP secretion compositions of the invention) comprise one or more receptor activating compounds and/or ligands that are specifically adapted to activate OR2B11 (referred to herein as “OR2B11 activating compounds”), whereby pharmacodynamic activity similar to that induced via activation of OR2W1 (discussed above) is induced.

According to the invention, the GIP secretion compositions (and GLP-1/GIP secretion compositions) of the invention can comprise one or more of the following OR2B11 activating compounds: spearmint oil, 2-ethyl-3,5-dimethylpyrazine, coumarin, dicyclohexyl disulfide, coffee difuran, quinoline, and cinnamaldehyde.

In a preferred embodiment, the OR2B11 activating compound comprises spearmint oil.

According to the invention, the mass of the noted OR2B11 activating compounds in a GIP secretion composition (and GLP-1/GIP secretion composition) of the invention can similarly comprise any of the aforementioned ex vivo masses and mass ranges.

According to the invention, the noted OR2B11 activating compounds can also comprise any of the aforementioned EC₅₀ concentrations and EC₅₀ concentration ranges in a GLP-1/PYY secretion composition (and GLP-1/GIP secretion composition) of the invention.

### OR2J3 Modulation

In some embodiments of the invention, the GIP secretion compositions of the invention (and GLP-1/GIP secretion compositions of the invention) comprise one or more receptor activating compounds and/or ligands that are specifically adapted to activate OR2J3 (referred to herein as “OR2J3 activating compounds”), whereby pharmacodynamic activity similar to that induced via activation of OR2W1 (discussed above) is induced.

According to the invention, the GIP secretion compositions of the invention (and GLP-1/GIP secretion compositions of the invention) can thus comprise one or more of the following OR2J3 activating compounds, which, as indicated above, are adapted to activate OR2J3, whereby secretion of GIP is similarly induced in vivo: cis-3-hexen-1-ol and cinnamaldehyde.

According to the invention, the mass of the noted OR2J3 activating compounds in a GIP secretion composition (and GLP-1/GIP secretion composition) of the invention can similarly comprise any of the aforementioned ex vivo masses and mass ranges.

According to the invention, the noted OR2J3 activating compounds can also comprise any of the aforementioned EC₅₀ concentrations and EC₅₀ concentration ranges in a GLP-1/PYY secretion composition (and GLP-1/GIP secretion composition) of the invention.

### TRPA1 Modulation

In some embodiments of the invention, the GIP secretion compositions of the invention (and GLP-1/GIP secretion compositions of the invention) comprise one or more receptor activating compounds and/or ligands that are adapted to modulate the activity of TRPA1, more particularly, activate TRPA1 (referred to herein as “TRPA1 activating compounds”), whereby a conformational change is induced in the molecular structure of TRPA1.

It is believed that when a compound/ligand, e.g., cinnamaldehyde, binds to TRPA1, the compound increases cellular Ca²⁺ and, thereby, increased serotonin (5-HT) secretion from endogenous enterochromaffin cells.

The serotonin secreted from the enterochromaffin cells binds to 5-HT receptors of endogenous gastrointestinal cells and, thereby, induces GIP secretion by the endogenous cells.

According to the invention, the GIP secretion compositions (and GLP-1/GIP secretion compositions) of the invention can comprise one or more of the following TRPA1 activating compounds, which, as indicated above, are adapted to activate TRPA1, whereby secretion of GIP is similarly induced in vivo: cinnamaldehyde, allyl isothiocyanate, farnesyl thiosalicylic acid, formalin, hydrogen peroxide, 4-hydroxynonenal, and acrolein.

In a preferred embodiment, the TRPA1 activating compound comprises cinnamaldehyde.

According to the invention, the mass of the noted TRPA1 activating compounds in a GIP secretion composition (and GLP-1/GIP secretion composition) of the invention can similarly comprise any of the aforementioned ex vivo masses and mass ranges.

According to the invention, the noted TRPA1 activating compounds can also comprise any of the aforementioned EC₅₀ concentrations and EC₅₀ concentration ranges in a GLP-1/PYY secretion composition (and GLP-1/GIP secretion composition) of the invention.

Preferably, the GIP secretion compositions of the invention (and GLP-1/GIP secretion compositions of the invention) are formulated and adapted to induce multiple receptor activity via activation of a plurality of olfactory receptors, e.g., OR51E1, OR2C1, and OR2W1, and a plurality of free fatty acid receptors, e.g., FFAR1 and FFAR4, and, in some instances, a plurality of transient receptor potential ion channels (e.g., TRPA1).

According to the invention, the GIP secretion compositions of the invention and, as discussed below, GLP-1/GIP secretion compositions of the invention, are adapted to induce at least 50% activation of at least FFAR1 and/or FFAR4 and/or OR2W1 and/or OR2B11 and/or OR2J3 and/or TRPA1 in vivo when delivered to a patient.

In a preferred embodiment, the GIP secretion compositions of the invention (and GLP-1/GIP secretion compositions of the invention) are adapted to induce at least 50% activation of FFAR1 or FFAR4 in vivo when delivered to a patient.

### Concomitant Modulation of GLP-1 and GIP Secretion

In some embodiments of the invention, the receptor activating compounds and ligands, and compositions of the invention formed therefrom are specifically adapted to bind to and activate at least one receptor that induces GLP-1 secretion and at least one receptor that induces GIP secretion in vivo, including, without limitation, OR51E1, OR1A1, OR2C1, OR10J5, OR2W1, OR2B11, OR2J3, FFAR1, FFAR4, and TRPA1, whereby GLP-1 and GIP secretion is induced in vivo (referred to herein as “GLP-1/GIP secretion compositions”).

According to the invention, GLP-1/GIP secretion compositions can thus comprise one or more of the receptor activating compounds/ligands set forth in Tables I and II and discussed above.

As indicated above, according to the invention, the mass of the receptor activating compounds/ligands contained in a GLP-1/GIP secretion composition of the invention can comprise any of the aforementioned ex vivo masses and mass ranges.

As also indicated above, the concentration of the receptor activating compounds/ligands contained in a GLP-1/GIP secretion composition of the invention can also comprise any of the aforementioned EC₅₀ concentrations and EC₅₀ concentration ranges.

In some embodiments, the GLP-1/GIP secretion compositions of the invention are specifically formulated and adapted to provide multiple receptor activity and, thereby, activate at least OR51E1 and FFAR1 in vivo when delivered to a patient.

In some embodiments, the GLP-1/GIP secretion compositions of the invention thus comprise eugenol and/or butyl butyryl lactate and lauric acid.

In some embodiments, the weight % (w/w) of the eugenol in the GLP-1/GIP secretion composition comprises in the range of approximately 0.1% to approximately 10.0% (w/w), more preferably, in the range of approximately 1.0% to approximately 3.0% (w/w).

In some embodiments, the weight % (w/w) of the eugenol in the GLP-1/GIP secretion composition comprises in the range of approximately 3.0% to approximately 5.0% (w/w).

In some embodiments, the weight % (w/w) of eugenol contained in the GLP-1/GIP secretion composition comprises at least approximately 1.0%.

In some embodiments, the weight % (w/w) of eugenol contained in the GLP-1/GIP secretion composition comprises at least approximately 3.0%.

In some embodiments, the EC₅₀ concentration of eugenol contained in the GLP-1/GIP secretion composition comprises at least approximately 0.1 μM.

In some embodiments, the EC₅₀ concentration of eugenol contained in the GLP-1/GIP secretion composition comprises in the range of approximately 0.1 μM to approximately 350.0 μM, more preferably, in the range of approximately 200.0 μM to approximately 300.0 μM.

In some embodiments, the weight % (w/w) of the butyl butyryl lactate in the GLP-1/GIP secretion composition comprises in the range of approximately 0.1% to approximately 10.0% (w/w), more preferably, in the range of approximately 1.0% to approximately 3.0% (w/w).

In some embodiments, the weight % (w/w) of the butyl butyryl lactate in the GLP-1/GIP secretion composition comprises in the range of approximately 3.0% to approximately 5.0% (w/w).

In some embodiments, the weight % (w/w) of butyl butyryl lactate contained in the GLP-1/GIP secretion composition comprises at least approximately 1.0%.

In some embodiments, the weight % (w/w) of butyl butyryl lactate contained in the GLP-1/GIP secretion composition comprises at least approximately 3.0%.

In some embodiments, the EC₅₀ concentration of butyl butyryl lactate contained in the GLP-1/GIP secretion composition comprises at least approximately 0.1 μM.

In some embodiments, the EC₅₀ concentration of butyl butyryl lactate contained in the GLP-1/GIP secretion composition comprises in the range of approximately 0.1 μM to approximately 250.0 μM, more preferably, in the range of approximately 150.0 μM to approximately 250.0 μM.

In some embodiments, the weight % (w/w) of the lauric acid in the GLP-1/GIP secretion composition comprises in the range of approximately 0.01% to approximately 5.0% (w/w), more preferably, in the range of approximately 0.05% to approximately 0.2% (w/w).

In some embodiments, the weight % (w/w) of the lauric acid in the GLP-1/GIP secretion composition comprises in the range of approximately 0.1% to approximately 0.5% (w/w).

In some embodiments, the weight % (w/w) of lauric acid contained in the GLP-1/GIP secretion composition comprises at least approximately 0.05%.

In some embodiments, the weight % (w/w) of lauric acid contained in the GLP-1/GIP secretion composition comprises at least approximately 0.1%.

In some embodiments, the EC₅₀ concentration of lauric acid contained in the GLP-1/GIP secretion composition comprises at least approximately 0.05 μM.

In some embodiments, the EC₅₀ concentration of lauric acid contained in the GLP-1/GIP secretion composition comprises in the range of approximately 0.05 μM to approximately 50.0 μM, more preferably, in the range of approximately 5.0 μM to approximately 25.0 μM.

In some embodiments, the GLP-1/GIP secretion compositions of the invention are also formulated and adapted to activate at least TRPA1 in vivo when delivered to a patient.

In some embodiments, the GLP-1/GIP secretion compositions of the invention thus comprise cinnamaldehyde and allyl isothiocyanate.

In a preferred embodiment, the TRPA1 activating compound comprises cinnamaldehyde.

In some embodiments, the weight % (w/w) of the cinnamaldehyde in the GLP-1/GIP secretion composition comprises in the range of approximately 1.0% to approximately 30.0% (w/w), more preferably, in the range of approximately 11.0% to approximately 13.5% (w/w).

In some embodiments, the weight % (w/w) of the cinnamaldehyde in the GLP-1/GIP secretion composition comprises in the range of approximately 24.0% to approximately 26.0% (w/w).

In some embodiments, the weight % (w/w) of cinnamaldehyde contained in the GLP-1/GIP secretion composition comprises at least approximately 11.0%.

In some embodiments, the weight % (w/w) of cinnamaldehyde contained in the GLP-1/GIP secretion composition comprises at least approximately 24.0%.

In some embodiments, the EC₅₀ concentration of cinnamaldehyde contained in the GLP-1/GIP secretion composition comprises at least approximately 0.1 μM.

In some embodiments, the EC₅₀ concentration of cinnamaldehyde contained in the GLP-1/GIP secretion composition comprises in the range of approximately 0.1 μM to approximately 3000.0 μM, more preferably, in the range of approximately 500.0 μM to approximately 2500 μM.

In some embodiments, the GLP-1/GIP secretion compositions of the invention are also formulated and adapted to activate at least OR2W1 in vivo when delivered to a patient.

In some embodiments, the GLP-1/GIP secretion compositions of the invention thus comprise benzyl acetate and (−)-citronellol.

In a preferred embodiment, the OR2W1 activating compound comprises benzyl acetate.

In some embodiments, the weight % (w/w) of the benzyl acetate in the GLP-1/GIP secretion composition comprises in the range of approximately 0.01% to approximately 5.0% (w/w), more preferably, in the range of approximately 0.1% to approximately 1.0% (w/w).

In some embodiments, the weight % (w/w) of the benzyl acetate in the GLP-1/GIP secretion composition comprises in the range of approximately 0.5% to approximately 2.0% (w/w).

In some embodiments, the weight % (w/w) of benzyl acetate contained in the GLP-1/GIP secretion composition comprises at least approximately 0.1%.

In some embodiments, the weight % (w/w) of benzyl acetate contained in the GLP-1/GIP secretion composition comprises at least approximately 0.5%.

In some embodiments, the EC₅₀ concentration of benzyl acetate contained in the GLP-1/GIP secretion composition comprises at least approximately 0.1 μM.

In some embodiments, the EC₅₀ concentration of benzyl acetate contained in the GLP-1/GIP secretion composition comprises in the range of approximately 0.1 μM to approximately 200.0 μM, more preferably, in the range of approximately 50.0 μM to approximately 150.0 μM.

In some embodiments, the GLP-1/GIP secretion compositions of the invention are also formulated and adapted to activate at least OR2B11 in vivo when delivered to a patient.

In a preferred embodiment, the OR2B11 activating compound comprises spearmint oil.

In some embodiments, the weight % (w/w) of the spearmint oil in the GLP-1/GIP secretion composition comprises in the range of approximately 0.1% to approximately 10.0% (w/w), more preferably, in the range of approximately 2.0% to approximately 5.0% (w/w).

In some embodiments, the weight % (w/w) of the spearmint oil in the GLP-1/GIP secretion composition comprises in the range of approximately 7.0% to approximately 9.0% (w/w).

In some embodiments, the weight % (w/w) of spearmint oil contained in the GLP-1/GIP secretion composition comprises at least approximately 2.0%.

In some embodiments, the weight % (w/w) of spearmint oil contained in the GLP-1/GIP secretion composition comprises at least approximately 7.0%.

In some embodiments, the EC₅₀ concentration of spearmint oil contained in the GLP-1/GIP secretion composition comprises at least approximately 0.1 μM.

In some embodiments, the EC₅₀ concentration of spearmint oil contained in the GLP-1/GIP secretion composition comprises in the range of approximately 0.1 μM to approximately 1000.0 μM, more preferably, in the range of approximately 100.0 μM to approximately 500.0 μM.

As indicated above, in a preferred embodiment, the GLP-1/GIP secretion compositions of the invention are adapted to induce at least 50% activation of at least OR51E1 and/or OR1A1 and/or OR2C1 and/or OR10J5 and/or FFAR1 and/or FFAR4 and/or OR2W1 and/or OR2B11 and/or OR2J3 and/or TRPA1 in vivo when delivered to a patient.

As also indicated above, in a preferred embodiment, the GLP-1/GIP secretion compositions of the invention are adapted to induce at least 50% activation of multiple receptors; particularly, at least OR51E1 and FFAR1 or FFAR4.

According to the invention, modulating the activity of multiple receptors, e.g., olfactory receptors and/or free fatty acid receptors and/or transient receptor potential ion channels, as described herein, results in elevated endocrine factor levels.

In some embodiments, modulating the activity of multiple receptors, e.g., olfactory receptors and/or free fatty acid receptors and/or transient receptor potential ion channels, as described herein, results in synergistically elevated endocrine factor levels.

In some embodiments, modulating the activity of multiple receptors, e.g., olfactory receptors and/or free fatty acid receptors and/or transient receptor potential ion channels, as described herein, results in elevated endocrine factor secretion.

In some embodiments, modulating the activity of multiple receptors, e.g., olfactory receptors and/or free fatty acid receptors and/or transient receptor potential ion channels, as described herein, results in synergistically elevated endocrine factor secretion.

In some embodiments, modulating the activity of multiple receptors, e.g., olfactory receptors and/or free fatty acid receptors and/or transient receptor potential ion channels, as described herein, results in endocrine factor secretion higher than endocrine factor secretion induced when modulating the activity of any single receptor alone.

In some embodiments, modulating the activity of multiple receptors, e.g., olfactory receptors and/or free fatty acid receptors and/or transient receptor potential ion channels, as described herein, induces a beneficial biological response, including, by way of example, increased insulin secretion, lower food consumption, increased body mass reduction, increased CAMP levels, increased nutrient absorption, increased small intestinal length, increased small intestinal weight, increased villus height, or increased villus height/crypt depth ratio than when modulating the activity of any single receptor alone.

In some embodiments, the compositions of the invention further comprise a physiologically suitable (or acceptable) carrier (also referred to herein as a physiologically suitable (or acceptable) excipient, or physiologically suitable (or acceptable) excipient selected based on a chosen route of administration, e.g., oral administration, and standard pharmaceutical practice.

According to the invention, suitable aqueous and non-aqueous carriers that can be employed in the compositions of the invention include water, ethanol, polyols (such as glycerol, glycerin-based water, propylene glycol, polyethylene glycol, and the like), and suitable mixtures thereof; vegetable oils, such as olive oil; oils derived from the seeds of plants, such as sunflower seed oil; buffers, such as neutral buffered saline, phosphate buffered saline and the like; carbohydrates, such as glucose, mannose, sucrose, and dextran, mannitol; proteins; polypeptides, and amino acids, such as glycine; antioxidants; chelating agents such as ethylenediaminetetraacetic acid (EDTA) or glutathione; adjuvants (e.g., aluminum hydroxide); and injectable organic esters, such as ethyl oleate and cyclodextrins.

According to the invention, the compositions can be manufactured by means of conventional mixing, dissolving, granulating, dragee-making, levigating, emulsifying, encapsulating, entrapping, and lyophilizing processes. The manufactured compositions can take the form of solutions, suspensions, emulsion, tablets, pills, pellets, capsules, capsules containing liquids, powders, sustained-release formulations, suppositories, emulsions, aerosols, sprays, suspensions, and other forms suitable for administration to a subject or patient.

In some embodiments, proper fluidity of a composition is maintained via coating materials, such as lecithin.

The compositions can also comprise formulation additives, such as suspending agents, preservatives, stabilizers and/or dispersants, and preservation agents.

According to the invention, the compositions can thus be administered to a patient via any suitable method, including, without limitation, oral, sublingual, inhalation, intranasal, epidural, intracerebral, transdermal, topical, and injection administration means.

Thus, as indicated above, in some embodiments, the compositions of the invention are formed in tablet or capsule form to accommodate oral delivery to a subject or patient.

According to the invention, the compositions of the invention can also be incorporated into various ingestible fluids, such as flavored waters and coffee.

According to the invention, the compositions of the invention can also be incorporated into a food item, such as a cracker, and/or a nutritional supplement or supplemental food item, such as protein bar.

According to the invention, the compositions of the invention can be administered at any of the following dosage ranges: from approximately 1.0 μg/kg to approximately 1.0 kg/kg, approximately 10.0 μg/kg to approximately 100.0 g/kg, approximately 10.0 μg/kg to approximately 25.0 mg/kg, approximately 100.0 μg/kg to approximately 50.0 g/kg, approximately 100.0 μg/kg to approximately 50.0 mg/kg, approximately 500.0 μg/kg to approximately 25.0 g/kg, approximately 500.0 μg/kg to approximately 100.0 mg/kg, approximately 1.0 mg/kg to approximately 10.0 g/kg, approximately 1.0 mg/kg to approximately 50.0 mg/kg, approximately 5.0 mg/kg to approximately 5.0 g/kg, approximately 5.0 mg/kg to approximately 25.0 mg/kg, approximately 10.0 mg/kg to approximately 2.5 g/kg, approximately 10.0 mg/kg to approximately 200.0 mg/kg, approximately 25.0 mg/kg to approximately 1.5 g/kg, approximately 25.0 mg/kg to approximately 750.0 mg/kg, approximately 50.0 mg/kg to approximately 1.0 g/kg, approximately 50.0 mg/kg to approximately 600.0 mg/kg, approximately 75.0 mg/kg to approximately 550.0 mg/kg, approximately 100.0 mg/kg to approximately 500.0 mg/kg, approximately 150.0 mg/kg to approximately 400.0 mg/kg, and approximately 200.0 mg/kg to approximately 350.0 mg/kg.

The compositions of the invention can also be administered at any dosage between the above referenced dosage ranges.

The compositions of the invention can thus also be administered at a dosage of at least approximately 1.0 μg/kg, at least approximately 10.0 μg/kg, at least approximately 100.0 μg/kg, at least approximately 500.0 μg/kg, at least approximately 1.0 mg/kg, at least approximately 5.0 mg/kg, at least approximately 10.0 mg/kg, at least approximately 25.0 mg/kg, at least approximately 50.0 mg/kg, at least approximately 75.0 mg/kg, at least approximately 100.0 mg/kg, at least approximately 150.0 mg/kg, and at least approximately 200.0 mg/kg.

According to the invention, the compositions of the invention can also be administered at one of the dosage ranges (and/or dosages therebetween and/or aforementioned ex vivo masses) over a prescribed time, by way of example, from approximately 1.0 μg to approximately 1.0 kg per day, from approximately 100.0 μg to approximately 500.0 g per day, from approximately 500.0 μg to approximately 100.0 g per day, from approximately 1.0 mg to approximately 20.0 g per day, from approximately 2.5 mg to approximately 15.0 g per day, from approximately 5.0 mg to approximately 10.0 g per day, from approximately 10.0 mg to approximately 5.0 g per day, from approximately 25.0 mg to approximately 2.5 g per day, from approximately 50.0 mg to approximately 2.0 g per day, from approximately 100.0 mg to approximately 1.5 g per day, from approximately 150.0 mg to approximately 1.0 g per day, from approximately 200.0 mg to approximately 750.0 mg per day, and from approximately 250.0 mg to approximately 500.0 mg per day.

In some embodiments, a composition; particularly, a GLP-1/GIP secretion composition of the invention, is administered to a subject in accordance with a dosage delivery protocol comprising first delivery of a first dose of the composition to the subject at a first time, second delivery of a second dose of the composition to the subject at a first period of time in the range of approximately 2.5 to approximately 3.5 hours after the first delivery of the first dose of the composition to the subject, third delivery of a third dose of the composition to the subject at a second period of time in the range of approximately 2.5 to approximately 3.5 hours after the second delivery of the second dose of the composition to the subject, and fourth delivery of a fourth dose of the composition to the subject at a third period of time in the range of approximately 2.5 to approximately 3.5 hours after the third delivery of the third dose of the composition to the subject.

In some embodiments, a composition; particularly, a GLP-1/GIP secretion composition of the invention, is administered to a subject in accordance with a dosage delivery protocol comprising first delivery of a first dose of the composition to the subject at a first time, second delivery of a second dose of the composition to the subject at a first period of time in the range of approximately 3.5 to approximately 4.5 hours after the first delivery of the first dose of the composition to the subject, third delivery of a third dose of the composition to the subject at a second period of time in the range of approximately 3.5 to approximately 4.5 hours after the second delivery of the second dose of the composition to the subject, and fourth delivery of a fourth dose of the composition to the subject at a third period of time in the range of approximately 3.5 to approximately 4.5 hours after the third delivery of the third dose of the composition to the subject.

According to the invention, the noted dosages and delivery protocols are sufficient to induce sustained (i.e., extended periods) of GLP-1, PYY and/or GIP secretion in vivo.

In some embodiments of the invention, there are thus provided compositions and methods for treating endocrine disorders of a subject that are associated with excessive and, hence, unacceptable body mass.

In some embodiments of the invention, a method for reducing body mass of a subject comprising the following steps is provided:
(a) providing a composition comprising (i) a delivery medium, (ii) at least a first natural receptor activating compound adapted to bind to and activate at least one first receptor selected from the group comprising olfactory receptor family 51 subfamily E member 1 (OR51E1) and olfactory receptor family 2 subfamily C member 1 (OR2C1), and (iii) at least a second natural receptor activating compound adapted to bind to and activate at least one second receptor selected from the group comprising free fatty acid receptor 1 (FFAR1) and free fatty acid receptor 4 (FFAR4),
the composition adapted to induce glucagon-like peptide-1 (GLP-1) and gastric inhibitory polypeptide (GIP) secretion, and, thereby, increased insulin secretion in vivo, when the composition is delivered to the subject; and
(b) delivering the composition to the subject, wherein the composition suppresses the appetite of the subject.

In a preferred embodiment, the first receptor activating compound comprises butyl butyryl lactate.

In a preferred embodiment, the butyl butyryl lactate comprises an EC₅₀ concentration of at least 0.1 μM in the composition.

In a preferred embodiment, the second receptor activating compound comprises a compound selected from the group consisting of a medium-chain free fatty acid, a long-chain free fatty acid and an omega-3 polyunsaturated fatty acid.

In a preferred embodiment, the second receptor activating compound comprises a medium-chain free fatty acid.

In a preferred embodiment, the medium-chain free fatty acid comprises lauric acid.

In a preferred embodiment, the lauric acid comprises an EC₅₀ concentration of at least 0.05 μM in the composition.

In some embodiments of the invention, the composition comprises at least a third receptor activating compound adapted to bind to and activate at least one third receptor selected from the group comprising olfactory receptor family 2 subfamily B member 11 (OR2B11), olfactory receptor family 2 subfamily J member 3 (OR2J3), and transient receptor potential cation channel subfamily A member 1 (TRPA1).

In a preferred embodiment, the third receptor activating compound comprises cinnamaldehyde.

In a preferred embodiment, the cinnamaldehyde comprises an EC₅₀ concentration of at least 0.1 μM in the composition.

In some embodiments of the invention, the composition comprises at least a fourth receptor activating compound adapted to bind to and activate olfactory receptor family 2 subfamily W member 1 (OR2W1).

In a preferred embodiment, the fourth receptor activating compound comprises benzyl acetate.

In a preferred embodiment, the benzyl acetate comprises an EC₅₀ concentration of at least 0.1 μM in the composition.

In some embodiments, the method for reducing body mass of a subject comprises the following steps:
(a) providing a composition comprising a delivery medium, a first receptor activating compound, a second receptor activating compound and a third receptor activating compound,
the composition comprising a mass in the range of approximately 1200.0 mg to approximately 1400.0 mg,
the delivery medium comprising in the range of approximately 55.0% to approximately 60.0% (w/w) of the composition,
the first receptor activating compound comprising butyl butyryl lactate, the butyl butyryl lactate comprising in the range of approximately 3.0% to approximately 5.0% (w/w) of the composition,
the second receptor activating compound comprising lauric acid, the lauric acid comprising in the range of approximately 0.1% to approximately 0.5% (w/w) of the composition,
the third receptor activating compound comprising cinnamaldehyde, the cinnamaldehyde comprising in the range of approximately 24.0% to approximately 26.0% (w/w) of the composition,
the composition adapted to induce activation of at least olfactory receptor family 51 subfamily E member 1 (OR51E1), free fatty acid receptor 1 (FFAR1) and transient receptor potential cation channel subfamily A member 1 (TRPA1) in vivo; and
(b) delivering the composition to the subject, wherein secretion of glucagon-like peptide-1 (GLP-1) and gastric inhibitory polypeptide (GIP) and, thereby, increased insulin secretion is induced.

In some embodiments, the composition further comprises a fourth receptor activating compound adapted to bind to and induce activation of at least OR51E1.

In some embodiments, the fourth receptor activating compound comprises eugenol.

In some embodiments, the eugenol comprises at least approximately 3.0% (w/w) of the composition.

In some embodiments, the composition further comprises a fifth receptor activating compound. adapted to bind to and induce activation of at least olfactory receptor family 2, subfamily W, member 1 (OR2W1).

In some embodiments, the fifth receptor activating compound comprises benzyl acetate.

In some embodiments, the benzyl acetate comprises at least approximately 0.5% (w/w) of the composition.

In some embodiments, the composition further comprises a sixth receptor activating compound adapted to bind to and activate olfactory receptor family 2 subfamily B member 11 (OR2B11).

In some embodiments, the sixth receptor activating compound comprises spearmint oil.

In some embodiments, the spearmint oil comprises at least approximately 7.0% (w/w) of the composition.

In some embodiments, the delivery medium comprises sunflower seed oil.

In a preferred embodiment, delivery of the composition to the subject comprises enteric sequential delivery of the composition to the subject via a dosage delivery protocol comprising first delivery of a first dose of the composition to the subject at a first time, second delivery of a second dose of the composition to the subject at a first period of time in the range of approximately 3.5 to approximately 4.5 hours after the first delivery of the first dose of the composition to the subject, third delivery of a third dose of the composition to the subject at a second period of time in the range of approximately 3.5 to approximately 4.5 hours after the second delivery of the second dose of the composition to the subject, and fourth delivery of a fourth dose of the composition to the subject at a third period of time in the range of approximately 3.5 to approximately 4.5 hours after the third delivery of the third dose of the composition to the subject, whereby at least activity of OR51E1, FFAR1 and TRPA1 is induced and sustained from the first time of the first delivery of the first dose of the composition to a fourth period of time in the range of approximately 3.5 to approximately 4.5 hours after the fourth delivery of the fourth dose of the composition to the subject.

In some embodiments, the method for reducing body mass of a subject comprises the following steps:
(a) providing a composition comprising a delivery medium, a first receptor activating compound, a second receptor activating compound and a third receptor activating compound, the composition comprising a mass in the range of approximately 600.0 mg to 800.0 mg,
the delivery medium comprising in the range of approximately 79.0% to approximately 85.0% (w/w) of the composition,
the first receptor activating compound comprising butyl butyryl lactate, the butyl butyryl lactate comprising in the range of approximately 1.0% to approximately 3.0% (w/w) of the composition,
the second receptor activating compound comprising lauric acid, the lauric acid comprising in the range of approximately 0.05% to approximately 0.2% (w/w) of the composition,
the third receptor activating compound comprising cinnamaldehyde, the cinnamaldehyde comprising in the range of approximately 11.0% to approximately 13.5% (w/w) of the composition,
the composition adapted to induce activation of at least olfactory receptor family 51 subfamily E member 1 (OR51E1), free fatty acid receptor 1 (FFAR1) and transient receptor potential cation channel subfamily A member 1 (TRPA1) in vivo; and
(b) delivering the composition to the subject, wherein secretion of glucagon-like peptide-1 (GLP-1) and gastric inhibitory polypeptide (GIP) and, thereby, increased insulin secretion is induced.

In some embodiments, the composition further comprises a fourth receptor activating compound adapted to bind to and induce activation of at least OR51E1.

In some embodiments, the fourth receptor activating compound comprises eugenol.

In some embodiments, the eugenol comprises at least 1.0% (w/w) of the composition.

In some embodiments, the composition further comprises a fifth receptor activating compound adapted to bind to and induce activation of at least olfactory receptor family 2, subfamily W, member 1 (OR2W1).

In some embodiments, the fifth receptor activating compound comprises benzyl acetate.

In some embodiments, the benzyl acetate comprises at least 0.1% (w/w) of the composition.

In some embodiments, the composition further comprises a sixth receptor activating compound adapted to bind to and activate olfactory receptor family 2 subfamily B member 11 (OR2B11).

In some embodiments, the sixth receptor activating compound comprises spearmint oil.

In some embodiments, the spearmint oil comprises at least 2.0% (w/w) of the composition.

In some embodiments, the delivery medium comprises sunflower seed oil.

In a preferred embodiment, delivery of the composition to the subject comprises enteric sequential delivery of the composition to the subject via a dosage delivery protocol comprising first delivery of a first dose of the composition to the subject at a first time, second delivery of a second dose of the composition to the subject at a first period of time in the range of approximately 2.5 to approximately 3.5 hours after the first delivery of the first dose of the composition to the subject, third delivery of a third dose of the composition to the subject at a second period of time in the range of approximately 2.5 to approximately 3.5 hours after the second delivery of the second dose of the composition to the subject, and fourth delivery of a fourth dose of the composition to the subject at a third period of time in the range of approximately 2.5 to approximately 3.5 hours after the third delivery of the third dose of the composition to the subject, whereby at least activity of OR51E1, FFAR1 and TRPA1 is induced and sustained from the first time of the first delivery of the first dose of the composition to a fourth period of time in the range of approximately 2.5 to approximately 3.5 hours after the fourth delivery of the fourth dose of the composition to the subject.

### EXAMPLES

The following examples are provided to enable those skilled in the art to more clearly understand the present invention. The examples should not be considered as limiting the scope of the invention, but merely as representative thereof.

### Example I

### Study of In Vitro OR51E1 and FFAR1 Activity Induced by Butyl Butyryl Lactate and Lauric Acid

The purpose of the following example was to assess the in vitro activity of OR51E1 and FFAR1 induced by butyl butyryl lactate (BBL) and lauric acid (LA), which, as indicated above, would be reflective of downstream GLP-1, GIP, and PYY secretion.

As discussed in detail below, the in vitro activity of BBL and LA was determined by measuring the response of human OR51E1 and FFAR1 receptors to BBL and LA in a CAMP activity assay.

### CAMP Activity Assay

The CAMP activity assay comprised HEK293T cells transfected with human OR51E1 receptors and HEK293T cells transfected with human FFAR1 receptors.

The HEK293T cells were also transfected with the DNA for the CAMP response element (CRE), which was coupled to a promoter and the gene for firefly luciferase (CRE luciferase). The HEK293T cells were also transfected with DNA for constitutively generated Renilla luciferase.

During the incubation step of the CAMP activity assay, a luciferase substrate (i.e., luciferin compound) was introduced to the transfected HEK293T cells, and firefly luciferase enzymes produced by the HEK293T cells converted luciferin to oxyluciferin and, thereby, produced detectable bioluminescence. The detectable bioluminescence was then measured using a conventional plate reader, which detected and measured the luminescence and provided the readout data for the cAMP activity assay.

### Results

### BBL Induced OR51E1 Activity

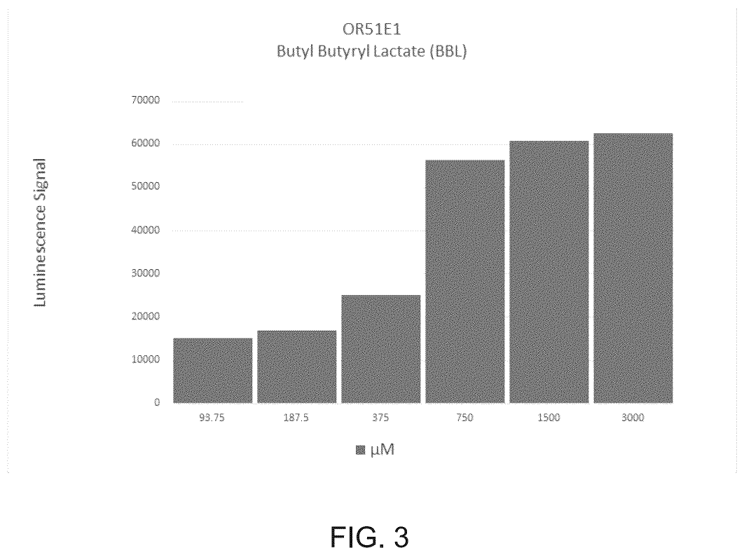

Referring now to FIG. 3, there is shown a bar graph reflecting the induced OR51E1 activity, expressed as luminescence values, by samples of BBL at concentrations of 93.75 μM, 187.5 μM, 375.0 μM, 750.0 μM, 1500.0 μM, and 3000.0 μM.

As reflected by FIG. 3, BBL at concentrations of 93.75 μM and above yielded significant levels of luminescence by the cells and, hence, unexpectedly marked inducement of OR51E1 activity in vitro.

Although prior studies by prominent researchers, including Saito, et al., Odor Coding by a Mammalian Receptor Repertoire, Science Signaling, vol. 2, no. 60 (2009): ra9-ra9, disclose that BBL can induce OR51E1 activity at EC₅₀ concentrations ≥1.0 mM, Applicant's study thus establishes that concentrations of BBL substantially lower than 1.0 mM (1000.0 μM), i.e., concentrations of BBL at 93.75 μM, 187.5 μM, 375.0 μM, and 750.0 μM, can and will induce significantly elevated levels OR51E1 activity in vitro.

Applicants' study thus not only confirms induced OR51E1 activity by BBL, but also unexpectedly reflects that BBL will induce significantly elevated levels OR51E1 activity in vitro at concentrations substantially lower than 1000.0 μM.

### LA Induced FFAR1 Activity

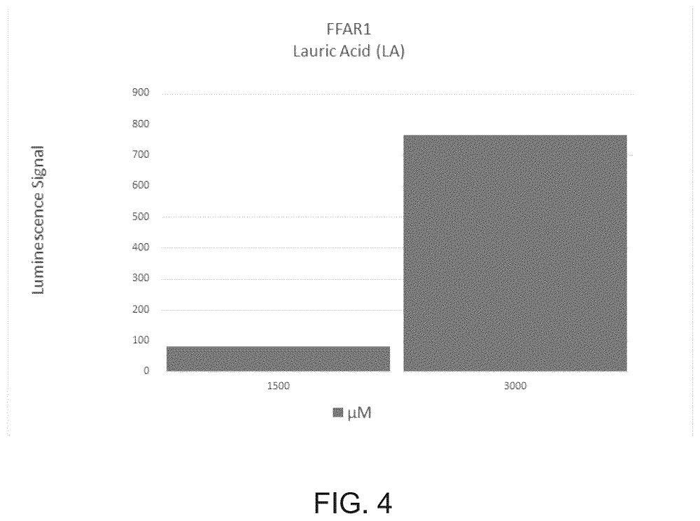

Referring now to FIG. 4, there is shown a bar graph reflecting induced FFAR1 activity, expressed as luminescence values, by samples of LA at concentrations of 1500.0 μM and 3000.0 μM.

As reflected by FIG. 4, LA at concentrations of 3000.0 μM and above yielded significant luminescence by the cells and, hence, significant inducement of FFAR1 activity in vitro.

In view of prior published studies³ of induced receptor activity by LA, it was anticipated that significant luminescence produced by the cells and, hence, significant levels of FFAR1 activity, would also have been exhibited by the cells in vitro at concentrations of ≤3000.0 μM. However, as discussed below, due to Applicant's test protocol; specifically, the use of a water-based buffer, a readable reliable luminescence signal was difficult to acquire.

As is well established, LA is virtually insoluble in water. Indeed, LA's solubility in water at 25° C. is 4.81 mg/L.

It is Applicants' position, which Applicants respectfully submit is in accord with established assay protocol(s), insolubility in a water-based buffer significantly limits the ability of the FFAR1 receptors of the cells to bind to the LA in the water-based buffer, which results in lower levels of luminescence produced by the cells. Difficulty detecting the luminescence produced by the cells at such low levels exasperates luminescence signal interference, such as background luminescence associated with non-specific binding and the laboratory environment.

It is further hypothesized by Applicants that it is possible that the basal or baseline behavior of the FFAR1 transfected cells in vitro also contributed to the difficulty acquiring a readable reliable luminescence signal at LA concentrations of ≤3000.0 μM.

Notwithstanding the low, and Applicants submit, inaccurate, luminescence signals at LA concentrations of ≤3000.0 μM reflected in FIG. 4, Applicants' study confirms, and FIG. 4 depicts, that LA can and will induce significant levels of FFAR1 activity, which is consistent with published studies.

Applicants further respectfully submit that it is highly likely that significant levels of FFAR1 activity would be exhibited by the cells at LA concentrations ≤3000.0 μM if LA was sufficiently solubilized in a specialized assay buffer to facilitate improved binding of the LA to the FFAR1 receptors of the cells.

### BBL & LA Mixture Induced FFAR1 Activity

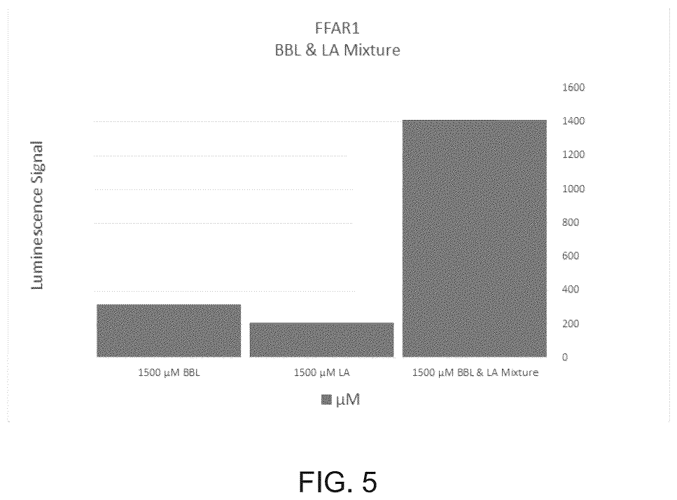

Referring now to FIG. 5, there is shown a bar graph reflecting the induced FFAR1 activity, expressed as luminescence values, by 1500.0 μM samples of BBL and LA, and a mixture of the BBL and LA.

Although BBL is not generally known as a FFAR1 ligand, as reflected in FIG. 5, when LA (a known FFAR1 ligand) is combined with LA, the BBL & LA mixture induces a level of luminescence that is markedly greater, i.e., approximately seven (7)-fold greater, than the luminescence induced by either BBL or LA alone, thus, evidencing unexpectedly enhanced, synergistic activity by and between BBL and LA, which results in significant inducement of FFAR1 activity.

Although the unexpectedly enhanced, synergistic activity by and between BBL and LA and, hence, significant inducement of FFAR1 activity resulting thereby was effectuated by a composition that included a 1:1 ratio of BBL and LA, it is respectfully submitted (and has been shown in preliminary unpublished studies) that the unexpectedly enhanced, synergistic activity by and between the BBL and LA could have and, hence, will also be effectuated at ratios of BBL to LA that fall within the weight percentages of BBL and LA in the compositions of the invention; particularly, compositions comprising in the range of approximately 1.0 to 3.0% (w/w) of BBL and in the range of approximately 0.05 to 0.2% (w/w) LA.

As also reflected by FIG. 5, although, as indicated above, BBL is not generally known as a FFAR1 ligand, BBL at a concentration of 1500.0 μM induced significant levels of luminescence by the cells and, hence, reflected an unexpectedly marked inducement of FFAR1 activity in vitro.

Contrary to published studies, it is thus highly likely that FFAR1 possesses functional binding affinity for BBL.

Applicants' study thus not only establishes induced FFAR1 activity by BBL alone, but also unexpectedly reflects enhanced, synergistic activity by and between BBL and LA at weight percentages in compositions of the invention, which results in significant inducement of FFAR1 activity.

### Eugenol Induced OR51E1 Activity

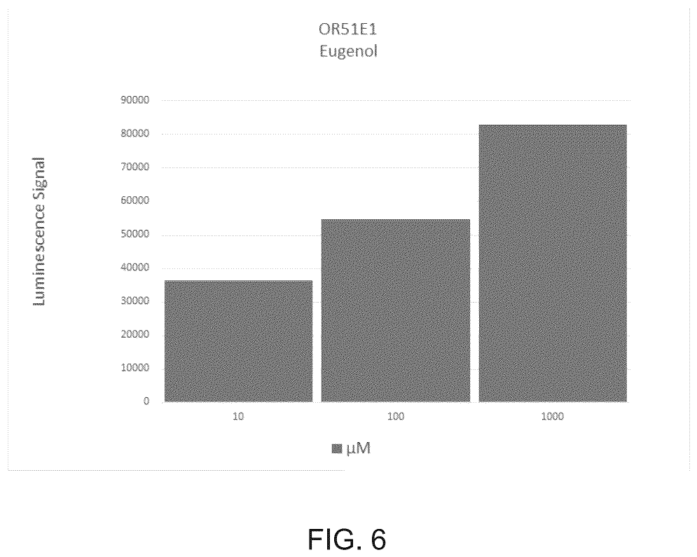

Referring now to FIG. 6, there is shown a bar graph reflecting the induced OR51E1 activity, expressed as luminescence values, by samples of eugenol at concentrations of 10.0 μM, 100.0 M, and 1000.0 μM.

As reflected by FIG. 6, eugenol at concentrations of 10.0 μM and above yielded significant levels of luminescence by the cells and, hence, unexpectedly marked inducement of OR51E1 activity in vitro.

Although prior studies by prominent researchers⁴ reflect that isoeugenol (i.e., an isomer of eugenol) and eugenol methyl ether (i.e., a eugenol derivative) are OR51E1 ligands, eugenol is not generally known as an OR51E1 ligand.

However, as reflected by FIG. 6, eugenol at concentrations of ≥10.0 μM induces significant levels of luminescence by the cells and, hence, unexpectedly marked inducement of OR51E1 activity in vitro. Contrary to published studies, it is thus highly likely that eugenol possesses functional binding affinity for OR51E1.

Applicants' study thus establishes induced OR51E1 activity by eugenol at weight percentages in compositions of the invention, which results in significant inducement of OR51E1 activity.

### Example II

### Evaluation of Physiological Changes Induced by a Composition of the Invention

### Objectives of Study

The purpose of the following example was to assess the physiological characteristics of individuals after administration of a composition of the invention. The physiological characteristics included body weight, body mass index (BMI), gastrointestinal function, cognitive and behavioral components associated with appetite, e.g., food consumption and noise, and tolerability.

### Study Participants

A total of one-hundred and twenty-five (125) individuals agreed to participate in the study. The individuals that participated in the study were initially screened to assess their eligibility to participate in the study. The inclusion criteria for the study participants comprised the following:
at least eighteen (18) years of age;
in good health, generally;
a self-reported BMI ≥25.0 kg/m² and ≤34.9 kg/m²;
a self-reported desire to lose weight;
has not taken any GLP-1 weight loss medications;
willing to refrain from taking any weight loss medications and supplements during the study; and
able to participate in the study for up to seventeen (17) weeks.

The exclusion criteria for the study comprised the following:
individuals that were on or planning to start a fad diet, keto diet, pure carnivore diet, raw food diet, fruitarian diet, or liquid diet;
individuals that were diagnosed with an alcohol use disorder and/or substance use disorder;
individuals currently pregnant, planning to become pregnant in the next one (1) month, or
individuals currently breastfeeding;
individuals having underlying medical conditions or comorbidities that could confound the evaluation of the study outcomes; and
individuals having known hypersensitivity or previous allergic reaction to cinnamaldehyde, spearmint oil, eugenol, butyl butyryl lactate, benzyl acetate, lauric acid, sodium-copper chlorophyllin, sunflower seed oil, gelatin, and/or glycerin.

### Composition of the Invention

The composition of the invention that was evaluated in the study comprised Applicants' commercial PIVIT™ composition comprising eugenol (˜2.0% (w/w)), butyl butyryl lactate (˜2.0% (w/w)), lauric acid (˜0.1% (w/w)), cinnamaldehyde (˜12.0% (w/w)), benzyl acetate (˜0.4% (w/w)) and spearmint oil (˜3.7% (w/w)).

The dosage protocol for the PIVIT™ composition consisted of enteric self-administration of four (4) PIVIT™ capsules, i.e., doses of PIVIT™ composition, daily at ˜2.5-hour to ˜3.5-hour intervals.

### Study Duration

The study was conducted over a total of seventeen (17) weeks, including the following study periods:
1) Screening and Recruitment period-four (4) weeks;
2) Baseline period-one (1) week; and
3) Product use period-twelve (12) weeks.

### Study Assessments

GSQ Assessment—The Gastrointestinal Symptom Questionnaire (GSQ) was employed to assess early-onset gastrointestinal side effects and tolerability of the PIVIT composition. It covered symptoms like nausea, heartburn, abdominal pain, and constipation.

TFEQ Assessment—The TFEQ Questionnaire, which is widely used in research studies related to eating disorders, obesity, and weight management, was employed to assess and monitor eating behaviors, such as binge eating.

Daily Assessments—Compliance with the PIVIT composition delivery protocol was assessed daily. A daily morning survey was also conducted to assess the participant's gastrointestinal symptoms, including stomach discomfort, constipation, bloating, and diarrhea.

Weekly Assessments—A weekly survey was also conducted to assess the participant's experience of “food noise”, lightheadedness, sweating, hunger, appetite, satiety and cravings. Each participant was also required to record his/her weight weekly.

Adverse Event Assessment—Each participant was also required to record any and all unusual symptoms, such as sleeping disorders, decreased energy, etc., when occurred. Each participant was also required to record any and all medical conditions that could confound the evaluation of the study outcomes, such as the common cold and flu.

Caloric Intake and Physical Activity Monitoring—Each participant was also encouraged to record all meals and physical activity, including duration and intensity levels.

### Results—Three (3) Weeks

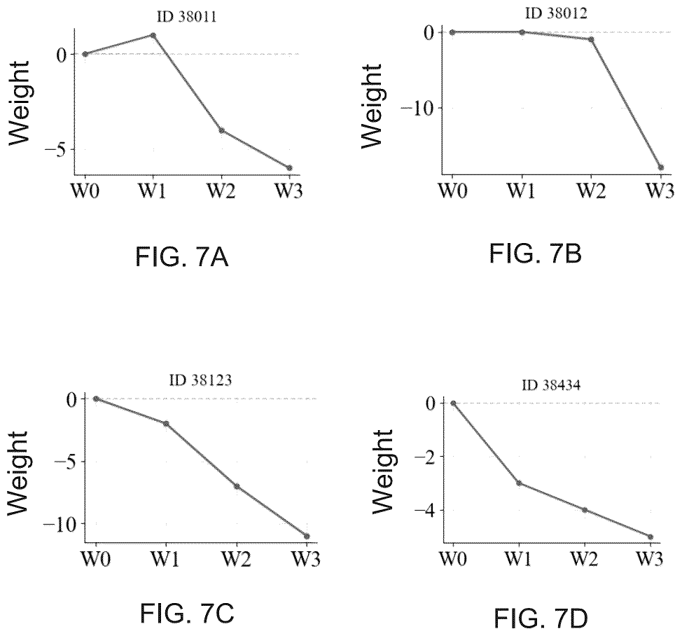

The results of the study through three (3) weeks are as follows:
The mean weight loss of the participants in the study was approximately 8.00 lbs. at the three (3) week time point. Graphs of four (4) participants depicting the substantial weight loss at the three (3) week time point are attached as FIGS. 7A-7D.
No gastrointestinal symptoms or issues or other physiological issues were reported.
Participants reported excellent tolerability of the PIVIT composition and reported no adverse effects associated therewith.
Participants reported significant reductions in appetite and “food noise”, and significantly increased feelings of satiety after meal consumption.
Participants also reported significantly reduced cravings for high calorie and calorically dense foods, and, in many instances, significantly reduced cravings for high calorie and calorically dense beverages, such as sweetened beverages and alcoholic beverages.
Participants also reported beneficial changes in eating habits, which included (i) significantly increased control over “food noise” in social situations, (ii) significantly increased control over alcohol intake in social situations, and (iii) significantly decreased instances of compulsive eating and drinking caused by both internal (e.g., psychological impulses) and external (e.g., societal pressures) factors.
Several participants even reported an increased dietary preference for nutrient dense foods, such as vegetables, legumes, whole grains, etc.

The study, even at a three (3) week point, demonstrates the effectiveness and safety of Applicants' PIVIT composition and, hence, product when taken in accordance with the recommended delivery protocol.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for reducing body mass of a subject, comprising the steps of:
providing a composition comprising a delivery medium, a first receptor activating compound, a second receptor activating compound and a third receptor activating compound,
said composition comprising a mass of about 1200.0 mg to about 1400.0 mg,
said delivery medium comprising in the range of about 55.0% to about 60.0% (w/w) of said composition,
said first receptor activating compound comprising butyl butyryl lactate, said butyl butyryl lactate comprising about 3.0% to about 5.0% (w/w) of said composition,
said second receptor activating compound comprising lauric acid, said lauric acid comprising about 0.1% to about 0.5% (w/w) of said composition,
said third receptor activating compound comprising cinnamaldehyde, said cinnamaldehyde comprising about 24.0% to about 26.0% (w/w) of said composition,
said composition adapted to induce activation of at least olfactory receptor family 51 subfamily E member 1 (OR51E1), free fatty acid receptor 1 (FFAR1) and transient receptor potential cation channel subfamily A member 1 (TRPA1) in vivo; and
delivering said composition to said subject, wherein secretion of glucagon-like peptide-1 (GLP-1) and gastric inhibitory polypeptide (GIP) is induced.

2. The method of claim 1, wherein said butyl butyryl lactate comprises at least 3.0% (w/w) of said composition.

3. The method of claim 1, wherein said lauric acid comprises at least 0.1% (w/w) of said composition.

4. The method of claim 1, wherein said cinnamaldehyde comprises at least 24.0% (w/w) of said composition.

5. The method of claim 1, wherein said composition further comprises a fourth receptor activating compound comprising eugenol, said eugenol comprising at least 3.0% (w/w) of said composition.

6. The method of claim 1, wherein said composition further comprises a fifth receptor activating compound comprising benzyl acetate, said benzyl acetate comprising at least 0.5% (w/w) of said composition.

7. The method of claim 1, wherein said composition further comprises a sixth receptor activating compound comprising spearmint oil, said spearmint oil comprises at least 7.0% (w/w) of said composition.

8. The method of claim 1, wherein said delivery medium comprises sunflower seed oil.

9. The method of claim 1, wherein said delivery of said composition to said subject comprises enteric sequential delivery of said composition to said subject via a dosage delivery protocol comprising first delivery of a first dose of said composition to said subject at a first time, second delivery of a second dose of said composition to said subject at a first period of time in the range of about 3.5 to about 4.5 hours after said first delivery of said first dose of said composition to said subject, third delivery of a third dose of said composition to said subject at a second period of time in the range of about 3.5 to about 4.5 hours after said second delivery of said second dose of said composition to said subject, and fourth delivery of a fourth dose of said composition to said subject at a third period of time in the range of about 3.5 to about 4.5 hours after said third delivery of said third dose of said composition to said subject, whereby said activation of said at least OR51E1, FFAR1 and TRPA1 is said induced and sustained from said first time of said first delivery of said first dose of said composition to a fourth period of time in the range of about 3.5 to about 4.5 hours after said fourth delivery of said fourth dose of said composition to said subject.

10. A method for reducing body mass of a subject, comprising the steps of:
providing a composition comprising a delivery medium, a first receptor activating compound, a second receptor activating compound and a third receptor activating compound,
said composition comprising a mass of about 600.0 mg to about 800.0 mg,
said delivery medium comprising in the range of about 79.0% to about 85.0% (w/w) of said composition,
said first receptor activating compound comprising butyl butyryl lactate, said butyl butyryl lactate comprising about 1.0% to about 3.0% (w/w) of said composition,
said second receptor activating compound comprising lauric acid, said lauric acid comprising about 0.05% to about 0.2% (w/w) of said composition,
said third receptor activating compound comprising cinnamaldehyde, said cinnamaldehyde comprising about 11.0% to about 13.5% (w/w) of said composition,
said composition adapted to induce activation of at least olfactory receptor family 51 subfamily E member 1 (OR51E1), free fatty acid receptor 1 (FFAR1) and transient receptor potential cation channel subfamily A member 1 (TRPA1) in vivo; and
delivering said composition to said subject, wherein secretion of glucagon-like peptide-1 (GLP-1) and gastric inhibitory polypeptide (GIP) is induced.

11. The method of claim 10, wherein said butyl butyryl lactate comprises at least 1.0% (w/w) of said composition.

12. The method of claim 10, wherein said lauric acid comprises at least 0.05% (w/w) of said composition.

13. The method of claim 10, wherein said cinnamaldehyde comprises at least 11.0% (w/w) of said composition.

14. The method of claim 10, wherein said composition further comprises a fourth receptor activating compound comprising eugenol, said eugenol comprising at least 1.0% (w/w) of said composition.

15. The method of claim 10, wherein said composition further comprises a fifth receptor activating compound comprising benzyl acetate, said benzyl acetate comprising at least 0.1% (w/w) of said composition.

16. The method of claim 10, wherein said composition further comprises a sixth receptor activating compound comprising spearmint oil, said spearmint oil comprises at least 2.0% (w/w) of said composition.

17. The method of claim 10, wherein said delivery medium comprises sunflower seed oil.

18. The method of claim 10, wherein said delivery of said composition to said subject comprises enteric sequential delivery of said composition to said subject via a dosage delivery protocol comprising first delivery of a first dose of said composition to said subject at a first time, second delivery of a second dose of said composition to said subject at a first period of time in the range of about 2.5 to about 3.5 hours after said first delivery of said first dose of said composition to said subject, third delivery of a third dose of said composition to said subject at a second period of time in the range of about 2.5 to about 3.5 hours after said second delivery of said second dose of said composition to said subject, and fourth delivery of a fourth dose of said composition to said subject at a third period of time in the range of about 2.5 to about 3.5 hours after said third delivery of said third dose of said composition to said subject, whereby said activation of said at least OR51E1, FFAR1 and TRPA1 is said induced and sustained from said first time of said first delivery of said first dose of said composition to a fourth period of time in the range of about 2.5 to about 3.5 hours after said fourth delivery of said fourth dose of said composition to said subject.
